(19) United States
(12) Patent Application Publication
(10) Pub. No.: US 2022/0184071 A1
(43) Pub. Date: Jun. 16, 2022

(54) GEPOTIDACIN FOR USE IN THE TREATMENT OF BACTERIAL URINARY TRACT INFECTIONS

(71) Applicant: GlaxoSmithKline Intellectual Property Development Limited, Brentford, Middlesex (GB)

(72) Inventors: David John PAYNE, Collegeville, PA (US); Stephen RITTENHOUSE, Collegeville, PA (US); Nicole SCANGARELLA-OMAN, Collegeville, PA (US)

(21) Appl. No.: 17/600,660

(22) PCT Filed: Apr. 3, 2019
(86) PCT No.: PCT/IB2020/000261

§ 371 Date: Oct. 1, 2021

Related U.S. Application Data
(60) Provisional application No. 62/828,801, filed on Apr. 3, 2019.

(51) Int. Cl. A61K 31/4985; A61P 31/04

(52) U.S. Cl. CPC A61K 31/4985; A61P 31/04

(57) ABSTRACT

Disclosed are methods and compounds for use for treating urinary tract infection comprising administering gepotidacin or a pharmaceutically acceptable salt thereof, in a therapeutically effective amount in a human in need thereof, wherein the urinary tract infection is caused by one or more bacterium as defined herein.

### CROSS-REFERENCE TO RELATED APPLICATIONS

This application claims benefit of U.S. 62/828,801, U.S. 62/834,112, U.S. 62/841,363, U.S. 62/895,594, U.S. 62/841,375, U.S. 62/895,590, U.S. 62/841,384 and U.S. 62/895,601, the disclosures of which are incorporated herein by reference in their entirety.

### STATEMENT REGARDING FEDERALLY SPONSORED RESEARCH

This invention was made with government support under the United States Of America Department Of Health And Human Services Assistant Secretary For Preparedness And Response, Biomedical Advanced Research and Development Authority (BARDA), within the Office of the Assistant Secretary for Preparedness and Response in the U.S. Department of Health and Human Services Agreement No.: HHSO100201300011C. The government has certain rights in this invention.

### FIELD OF THE PRESENT INVENTION

The present invention relates to methods and/or uses for treating bacterial infections caused by gram-positive and gram-negative bacteria, which comprises administering gepotidacin or pharmaceutically acceptable salts thereof, and/or corresponding pharmaceutical compositions as described herein.

### BACKGROUND OF THE PRESENT INVENTION

Over the past several decades, the frequency of antimicrobial resistance and its association with serious infectious diseases has increased at alarming rates.

For example, in the United States, the Centers for Disease Control and Prevention estimated that roughly 1.7 million hospital-associated infections, from all types of microorganisms, including bacteria, combined, cause or contribute to 99,000 deaths each year.

Infections caused by multidrug-resistant gram-positive and gram-negative bacteria represent a major public health burden, not just in terms of morbidity and mortality, but also in terms of increased expenditure on patient management and implementation of infection control measures. For example, in Europe, where hospital surveys have been conducted, the category of gram-negative infections are estimated to account for two-thirds of the 25,000 deaths each year. Nosocomial infections can cause severe pneumonia and infections of the urinary tract, bloodstream and other parts of the body. Many types are difficult to attack with antibiotics, and antibiotic resistance is spreading to gram-negative bacteria that can infect people outside the hospital (see, Pollack, Andrew. “Rising Threat of Infections Unfazed by Antibiotics” New York Times, Feb. 27, 2010). This high rate of resistance increases the morbidity, mortality, and costs associated with nosocomial infections.

Urinary tract infections (UTIs) are very common, with approximately 11% of women above the age of 18 years of age experiencing at least 1 episode of acute cystitis per year. Of these, half will experience more than 1 recurrent episode over their lifetime. The peak incidence occurs in young, sexually active women ages 18 to 29 years. UTIs may be caused by a variety of uropathogens; the predominant uropathogens isolated in community-acquired UTIs are Escherichia coli(75% to 90%) and Staphylococcus saprophyticus (5% to 15%), but other bacteria have also been reported.

New and effective oral antibiotic treatment options for UTI are needed, as therapies are becoming limited due to the increase of multidrug-resistant pathogens and extended-spectrum beta-lactamase-producing Enterobacteriaceae pathogens, which are impacting the efficacy of currently available oral antibacterial treatment options. Patient allergies or tolerances to certain antibiotics must also be taken into account in deciding on a treatment course.

To date, a variety of antibacterial drugs have been developed which have become clinically extremely important antimicrobial drugs. Researchers at GlaxoSmithKline described a novel class of antibacterial agents that target type IIA topoisomerases [see Nature, Volume 466, pages 935-940 (19 Aug. 2010) and Gibson et al. Mechanistic and Structural Basis for the Actions of the Antibacterial Gepotidacin against Staphylococcus aureus Gyrase, ACS Infectious Disease, 2019, 5, 570-581] that has shown activity against a broad spectrum of gram-positive and gram-negative bacteria. International Patent Publication WO 2008/128942 and U.S. Pat. No. 8,389,524, hereby incorporated by reference in their entirety, disclose tricyclic nitrogen containing compounds as antibacterial compounds, pharmaceutical compositions and corresponding uses thereof.

Thus, there is a demand for development of new antibiotic compounds which exhibit more potent antimicrobial activity with novel mechanisms of action and in particular, for corresponding methods and/or uses for treating certain bacterial infections caused by certain gram-positive and gram-negative bacterial aerobes and anaerobes.

### SUMMARY OF THE PRESENT INVENTION

In the first aspect, the present invention provides a method for treating urinary tract infection (UTI) comprising administering gepotidacin or a pharmaceutically acceptable salt thereof, in a therapeutically effective amount in a human in need thereof, wherein the UTI is caused by one or more bacterium selected from: Staphylococcus saprophyticus; Acinetobacter baumanni, Acinetobacter baumannii anitratus, Acinetobacter pittii, Citrobacter freundii complex, Citrobacter koseri, Haemophilus parainfluenzae, Haemophilus paraphrophilus, Klebsiella oxytoca, Klebsiella variicola, Leclercia adecarboxylata, Proteus hauseri, Proteus peneri, Serratia marcescens, Shigella boydii, Shigella flexneri, Shigella sonnei, Morganella morganii, Providencia rettgeri, drug-resistant Klebsiella pneumoniae, drug-resistant Escherichia coli, Acidovorax temperans, Citrobacter amalonaticus, Providencia stuartii, Pseudomonas putida;

Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans, Staphylococcus warneri, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massiliensis, Streptococcus mitis, Streptococcus oralis, Streptococcus mutans, Streptococcus parasanguinis, Streptococcus salivarius, Streptococcus sanguinis, Streptococcus vestibularis;

Bilophila wadsworthia, Sutterella wadsworthensis, Clostridium bifermentans, Clostridium difficile, Eggethella lenta, Peptostreptococcus anaerobius, Peptostreptococcus anaerobius, Bacteroides caccae, Bacteroides fragilis, Bacteroides ovatus, Bacteroides stercoris, Bacteroides thetaiotaomicron, Bacteroides uniformis, Bacteroides vulgatus, Fusobacterium necrophorum, Fusobacterium nucleatum, Porphyromonas asaccharolytica, Porphyromonas endodontalis, Porphyromonas gingivalis, Porphyromonas levii, Porphyromonas somerae, Prevotella bivia, Prevotella buccae, Prevotella denticola, Prevotella disiens, Prevotella melaninogenica, Veillonella alcalescens dispar, Veillonella parvula, Bifidobacterium adolescentis, Bifidobacterium breve, Bifidobacterium dentium, Bifidobacterium longum, Bifidobacterium pseudocatenulatum, Collinsella (Eubacterium) aerofaciens, Eubacterium limosum, Eubacterium nodatum, Lactobacillus acidophilus, Lactobacillus crispatus, Lactobacillus fermentum, Lactobacillus gasseri, Lactobacillus iners, Lactobacillus jensenii, Lactobacillus plantarum, and Lactobacillus rhamnosus.

In another aspect, the present invention provides a method for treating urinary tract infection (UTI) in a human comprising the following steps:

a) determining whether a sample from a human suspected of having UTI contains one or more bacterium selected from:

Staphylococcus saprophyticus; Acinetobacter baumannii, Acinetobacter baumannii anitratus, Acinetobacter pittii, Citrobacter freundii complex, Citrobacter koseri Haemophilus parainfluenzae, Haemophilus paraphrophilus, Klebsiella oxytoca, Klebsiella variicola, Leclercia adecarboxylata, Proteus hauseri, Proteus peneri, Serratia marcescens, Shigella boydii, Shigella flexneri, Shigella sonnei, Morganella morganii, Providencia rettgeri, drug-resistant Klebsiella pneumoniae, drug-resistant Escherichia coli, Acidovorax temperans, Citrobacter amalonaticus, Providencia stuartii, Pseudomonas putida;

Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans, Staphylococcus warneri, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massiliensis, Streptococcus mitis, Streptococcus oralis, Streptococcus mutans, Streptococcus parasanguinis, Streptococcus salivarius, Streptococcus sanguinis, Streptococcus vestibularis,

Bilophila wadsworthia, Sutterella wadsworthensis, Clostridium bifermentans, Clostridium difficile, Eggethella lenta, Peptostreptococcus anaerobius, Peptostreptococcus anaerobius, Bacteroides caccae, Bacteroides fragilis, Bacteroides ovatus, Bacteroides stercoris, Bacteroides thetaiotaomicron, Bacteroides uniformis, Bacteroides vulgatus, Fusobacterium necrophorum, Fusobacterium nucleatum, Porphyromonas asaccharolytica, Porphyromonas endodontalis, Porphyromonas gingivalis, Porphyromonas levii, Porphyromonas somerae, Prevotella bivia, Prevotella buccae, Prevotella denticola, Prevotella disiens, Prevotella melaninogenica, Veillonella alcalescens dispar Veillonella parvula, Bifidobacterium adolescentis, Bifidobacterium breve, Bifidobacterium dentium, Bifidobacterium longum, Bifidobacterium pseudocatenulatum, Collinsella (Eubacterium) aerofaciens, Eubacterium limosum, Eubacterium nodatum, Lactobacillus acidophilus, Lactobacillus crispatus, Lactobacillus fermentum, Lactobacillus gasseri, Lactobacillus iners, Lactobacillus jensenii, Lactobacillus plantarum, or Lactobacillus rhamnosus;

b) administering gepotidacin or a pharmaceutically acceptable salt thereof in a therapeutically effective amount to the subject if one or more of the bacterium is identified in the sample in step (a) and is determined to be the cause of the UTI.

In another aspect, the present invention provides gepotidacin or a pharmaceutically acceptable salt thereof for use in the treatment of UTI, wherein the UTI is caused by one or more bacterium selected from:

Staphylococcus saprophyticus; Acinetobacter baumannii, Acinetobacter baumannii anitratus, Acinetobacter pittii, Citrobacter freundii complex, Citrobacter koseri, Haemophilus parainfluenzae, Haemophilus paraphrophilus, Klebsiella oxytoca, Klebsiella variicola, Leclercia adecarboxylata, Proteus hauseri, Proteus peneri, Serratia marcescens, Shigella boydii Shigella flexneri, Shigella sonnei, Morganella morganii, Providencia rettgeri, drug-resistant Klebsiella pneumoniae, drug-resistant Escherichia coli Acidovorax temperans, Citrobacter amalonaticus, Providencia stuartii, Pseudomonas putida;

Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans, Staphylococcus warneri, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massiliensis, Streptococcus mitis, Streptococcus oralis, Streptococcus mutans, Streptococcus parasanguinis, Streptococcus salivarius, Streptococcus sanguinis, Streptococcus vestibularis;

Bilophila wadsworthia, Sutterella wadsworthensis, Clostridium bifermentans, Clostridium difficile, Eggethella lenta, Peptostreptococcus anaerobius, Peptostreptococcus anaerobius, Bacteroides caccae, Bacteroides fragilis, Bacteroides ovatus, Bacteroides stercoris, Bacteroides thetaiotaomicron, Bacteroides uniformis, Bacteroides vulgatus, Fusobacterium necrophorum, Fusobacterium nucleatum, Porphyromonas asaccharolytica, Porphyromonas endodontalis Porphyromonas gingivalis, Porphyromonas levii, Porphyromonas somerae, Prevotella bivia, Prevotella buccae, Prevotella denticola, Prevotella disiens, Prevotella melaninogenica, Veillonella alcalescens dispar Veillonella parvula, Bifidobacterium adolescentis, Bifidobacterium breve, Bifidobacterium dentium, Bifidobacterium longum, Bifidobacterium pseudocatenulatum, Collinsella (Eubacterium) aerofaciens, Eubacterium limosum, Eubacterium nodatum, Lactobacillus acidophilus, Lactobacillus crispatus, Lactobacillus fermentum, Lactobacillus gasseri, Lactobacillus iners, Lactobacillus jensenii, Lactobacillus plantarum, and Lactobacillus rhamnosus.

In another aspect, the present invention provides use of gepotidacin or a pharmaceutically acceptable salt thereof in the manufacture of a medicament for use in the treatment of UTI, wherein the UTI is caused by one or more bacterium selected from:

Staphylococcus saprophyticus; Acinetobacter baumannii, Acinetobacter baumannii anitratus, Acinetobacter pittii, Citrobacter freundii complex, Citrobacter koseri; Haemophilus parainfluenzae, Haemophilus paraphrophilus, Klebsiella oxytoca, Klebsiella variicola, Leclercia adecarboxylata, Proteus hauseri, Proteus peneri, Serratia marcescens, Shigella boydii, Shigella flexneri Shigella sonnei, Morganella morganii, Providencia rettgeri, drug-resistant Klebsiella pneumoniae, drug-resistant Escherichia coli Acidovorax temperans, Citrobacter amalonaticus, Providencia stuartii, Pseudomonas putida;

Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans, Staphylococcus warneri, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massiliensis, Streptococcus mitis, Streptococcus oralis, Streptococcus mutans, Streptococcus parasanguinis, Streptococcus salivarius, Streptococcus sanguinis, Streptococcus vestibularis;

Bilophila wadsworthia, Sutterella wadsworthensis, Clostridium bifermentans, Clostridium difficile, Eggethella lenta, Peptostreptococcus anaerobius, Peptostreptococcus anaerobius, Bacteroides caccae, Bacteroides fragilis, Bacteroides ovatus, Bacteroides stercoris, Bacteroides thetaiotaomicron, Bacteroides uniformis, Bacteroides vulgatus, Fusobacterium necrophorum, Fusobacterium nucleatum, Porphyromonas asaccharolytica, Porphyromonas endodontalis, Porphyromonas gingivalis, Porphyromonas levii, Porphyromonas somerae, Prevotella bivia, Prevotella buccae, Prevotella denticola, Prevotella disiens, Prevotella melaninogenica, Veillonella alcalescens dispar, Veillonella parvula, Bifidobacterium adolescentis, Bifidobacterium breve, Bifidobacterium dentium, Bifidobacterium longum, Bifidobacterium pseudocatenulatum, Collinsella (Eubacterium) aerofaciens, Eubacterium limosum, Eubacterium nodatum, Lactobacillus acidophilus, lactobacillus crispatus, Lactobacillus fermentum, lactobacillus gasser, Lactobacillus, Lactobacillus jensenii, Lactobacillus plantarum, and Lactobacillus rhamnosus.

### DESCRIPTION OF DRAWINGS/FIGURES

FIG. 1 shows the participant disposition and study outline of the phase ha clinical trial described in Example 3. BID=twice daily, PK=pharmacokinetic; TOC=test of cure; WBC=white blood cells.

FIG. 2 shows the baseline algorithm for the microbiological intent-to-treat population selection, as described in Example 3. CFU=colony-forming units; micro-ITT=microbiological intent-to-treat.

FIG. 3 shows the quantitative bacterial counts (CFU/mL) by baseline qualifying uropathogen over time (micro-ITT population), described in Example 3.

FIG. 4 shows the individual clinical symptom score and boxplot of total score over time (ITT population), described in Example 3.

FIG. 5 shows the gepotidacin median CT plasma concentration by day following BID oral administration of gepotidacin (1500 mg), as described in Example 3.

FIG. 6 shows the gepotidacin median plasma concentration-time profiles following single and BID oral administration (1500 mg), as described in Example 3.

FIG. 7 shows the median urine concentration time profiles following single and BID oral administration of gepotidacin (1500 mg), as described in Example 3.

FIG. 8 shows the plasma, kidney and thigh concentration vs. time profiles in the murine pyelonephritis and thigh infection models, as described in Example 4.

FIG. 9 shows Dependent Variable versus Prediction, and Conditionally Weighted Residuals versus Time and Prediction, as described in Example 4. DV=Dependent Variable, CWRES=Conditionally Weighted Residuals, PRED=Prediction.

FIG. 10 shows the correlation between efficacy and PK/PD indices (fAUC and fCmax) in the neutropenic thigh model, as described in Example 4.

### DETAILED DESCRIPTION OF THE PRESENT INVENTION

As used herein, the term “antibiotic” is synonymous with “antibacterial” and “antimicrobial”.

Gepotidacin is a first-in-class, triazaacenaphthylene antibiotic with the unique ability to selectively inhibit bacterial DNA replication by a means not utilized by any currently approved human therapeutic agent, therefore providing the opportunity to address an unmet medical need. Gepotidacin and its racemic form are disclosed in WO 2008/1289422. Gepotidacin is (2R)-2-({4-[(3,4-dihydro-2H-pyrano[2,3-c]pyridin-6-ylmethyl)amino]-1-piperidinyl}methyl)-1,2-dihydro-3H,8H-2a,5,8a-triazaacenaphthylene-3,8-dione:

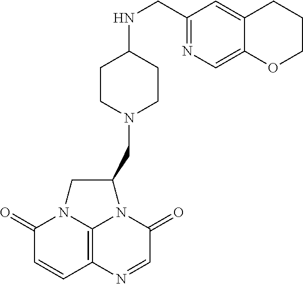 [SMILES: O=c1ccc2ncc(=O)n3c2n1C[C@H]3CN1CCC(NCc2cc3c(cn2)OCCC3)CC1]

As used herein, the term “gepotidacin” may mean gepotidacin free base, or a salt of gepotidacin. When a composition contains a salt of gepotidacin, the stated amount of gepotidacin in the composition refers to the amount of corresponding gepotidacin free base.

Gepotidacin has now been found to be surprisingly active against certain bacteria against which it has not previously been shown to have activity. Its unique mode of action means it can offer an alternative treatment to conventional antibiotics for certain bacterial infections. Further, gepotidacin has now been found to be particularly suited to treat UTI, because of its specific efficacy against certain bacteria which cause UTI, its in vivo safety profile, and the fact that its activity is minimally affected by urine. Moreover, gepotidacin has been shown to maintain a high and sustained concentration in the urine in vivo (see Examples herein). These unexpected properties of gepotidacin make it highly suitable for the use in the treatment of UTI.

In the first aspect, the present invention provides a method for treating UTI comprising administering gepotidacin or a pharmaceutically acceptable salt thereof, in a therapeutically effective amount in a human in need thereof, wherein the UTI is caused by one or more bacterium as described herein.

As used herein, the phrase “caused by a bacterium” may mean (1) that the skilled person suspects the identity of the bacterium that is causing the episode of UTI, for example due to patient history or local epidemiology; or (2) that the skilled person proves or determines the identity of the causative bacterium using culture (or other diagnostic test) information obtained from the infected patient. Thus in one embodiment, the present invention provides a method for treating UTI comprising administering gepotidacin or a pharmaceutically acceptable salt thereof, in a therapeutically effective amount in a human in need thereof, wherein the UTI is proven to be caused by one or more bacterium as described herein. In another embodiment, the present invention provides a method for treating UTI comprising administering gepotidacin or a pharmaceutically acceptable salt thereof, in a therapeutically effective amount in a human in need thereof, wherein the UTI is suspected or strongly suspected to be caused by one or more bacterium as defined herein.

In one embodiment, the present invention provides a method for treating UTI comprising administering gepotidacin or a pharmaceutically acceptable salt thereof, in a therapeutically effective amount in a human in need thereof, wherein the UTI is caused by a strain of bacterium as described herein, which is susceptible to gepotidacin or a pharmaceutically acceptable salt thereof.

As would be understood by the skilled person, “susceptible” means that an isolate of a microorganism is inhibited by the usually achievable concentration of an antimicrobial agent when the recommended dosage is used for the site of infection. Susceptibility to gepotidacin may be determined by the skilled person from an isolate recovered from a sample from an infected subject, using standard methods as published by e.g. the United States Federal Drug Agency (Antibacterial Susceptibility Test Interpretive Criteria), Clinical and Laboratory Standards Institute (for example Performance Standards for Antimicrobial Susceptibility Testing. 29th ed. CLSI supplement M100. Wayne, Pa.: Clinical and Laboratory Standards Institute; 2019) or the European Union Committee on Antimicrobial Susceptibility Testing.

In one embodiment, as used herein, “susceptible to gepotidacin” or “gepotidacin-susceptible” means that the gepotidacin MIC (which may be measured in vitro or in vivo) for an isolate of a bacterium is 32 mg/L or less, as measured by broth microdilution according to Clinical and Laboratory Standards Institute guidelines. In one embodiment, it means 16 mg/L or less. In one embodiment, it means 8 mg/mL or less. In one embodiment, it means 4 mg/L or less. In another embodiment, it means 2 mg/L or less. In another embodiment, it means 1 mg/L or less.

In one embodiment, prior to the administration of gepotidacin or a pharmaceutically acceptable salt thereof, the UTI is determined to be caused by one or more bacterium as defined above. The determination may be done by any conventional means. For example, a sample such as a urine or plasma sample may be obtained from the human suspected of having UTI, which is then tested for the presence of one or more bacterium as defined in the present invention, using any conventional means. If this culture information reveals the presence of one or more bacterium as defined in the present invention and is determined to be the cause of the UTI according to known diagnostic criteria, gepotidacin or a pharmaceutically acceptable salt thereof is administered in a therapeutically effective amount. A skilled person may use established diagnostic criteria, such as a bacterial count of more than 10³ CFU/mL, 10⁴ CFU/mL or 10⁵ CFU/mL of uropathogens in a mid-stream sample of urine (see for example Kunin C. Urinary tract infections, in detection, prevention and management. 1997, Lea & Febiger; European Association of Urology, Guidelines on Urological Infections 2015), to determine the causative bacterium of an episode of UTI.

In one embodiment, the present invention provides a method for treating cystitis caused by Staphylococcus saprophyticus, which comprises administering gepotidacin or a pharmaceutically acceptable salt thereof to a patient in need thereof. In one embodiment, the cystitis is acute cystitis.

In the second aspect, the present invention provides a method for treating urinary tract infection (UTI) comprising the steps of (a) determining whether a sample from a human suspected of having UTI contains one or more bacterium as defined in the present invention, and (b) administering gepotidacin or a pharmaceutically acceptable salt thereof in a therapeutically effective amount to the subject if one or more of the bacterium is identified in the sample in step (a) and is determined to be the cause of the UTI. The determination of the presence of the bacterium of the present invention in the sample may be done using any conventional means, as described above.

In one embodiment, the sample is used in step (a) is a urine sample. In another embodiment, the sample is a blood, plasma or tissue sample.

In the third aspect, the present invention provides gepotidacin or a pharmaceutically acceptable salt thereof for use in the treatment of UTI, wherein the UTI is caused by one or more bacterium as defined above.

In another aspect, the present invention provides use of gepotidacin or pharmaceutically acceptable salt thereof for treatment of urinary tract infection caused by a gram-positive aerobe organism or a gram-negative aerobe organism;

wherein:

the gram-positive aerobe organism is Staphylococcus saprphyticus;

the gram-negative aerobe organism is selected from Acinetobacter baumannii anitratus, Acinetobacter pittii; Haemophilus parainfluenzae, Haemophilus paraphrophilus, Klebsiella oxytoca, Klebsiella variicola, Proteus hauseri or Proteus peneri.

In one embodiment, in any of the aspects of the invention, the bacterium is selected from: Staphylococcus saprophyticus; Acinetobacter baumannii, Acinetobacter baumanni/anitratus, Acinetobacter pittii, Citrobacter freundii complex, Citrobacter koseri, Haemophilus parainfluenzae, Haemophilus paraphrophilus., Klebsiella oxytoca, Klebsiella variicola, Leclercia adecarboxylata, Proteus hauseri, Proteus peneri, Serratia marcescens, Shigella boydii, Shigella flexneri, Shigella sonnei, Morganella morganii, Providencia rettgeri, drug-resistant Klebsiella pneumoniae, drug-resistant Escherichia coli, Acidovorax temperans, Citrobacter amalonaticus, Providencia stuartii and Pseudomonas putida.

In one embodiment, in any of the aspects of the invention, the bacterium is selected from: Staphylococcus saprophyticus, Acinetobacter baumannii anitratus, Acinetobacter pitti, Citrobacter freundii complex, Citrobacter koseri, Haemophilus parainfluenzae, Haemophilus paraphrophilus, Klebsiella oxytoca, Klebsiella variicola, Leclercia adecarboxylata, Proteus hauseri Proteus peneri, Morganella morganii, Providencia rettgeri, and Serratia marcescens.

In one embodiment, in any of the aspects of the invention, the bacterium is selected from: Staphylococcus saprophyticus, drug-resistant Staphylococcus saprophyticus, Acinetobacter pittii, Citrobacter freundii complex, Citrobacter koseri, drug-resistant Citrobacter koseri, Klebsiella oxytoca, Klebsiella variicola, Proteus hauseri Proteus peneri, Serratia marcescens, drug-resistant Klebsiella pneumoniae, and drug-resistant Escherichia coli.

In one embodiment, in any of the aspects of the invention, the bacterium is selected from:

Staphylococcus saprophyticus, drug-resistant Staphylococcus saprophyticus, Proteus hauseri, Proteus peneri, drug-resistant Klebsiella pneumoniae, and drug-resistant Escherichia coli.

In one embodiment, in any of the aspects of the invention, the bacterium is selected from: Staphylococcus saprophyticus and drug-resistant Staphylococcus saprphyticus.

In one embodiment, in any of the aspects of the invention, the bacterium is Staphylococcus saprophyticus.

In one embodiment, in any of the aspects of the invention, the bacterium is Morganella morganii or Providencia rettgeri.

In one embodiment, the bacterium is non-drug resistant or drug resistant Morganella morganii.

In one embodiment, the bacterium is non-drug resistant or drug resistant Providencia rettgeri.

In one embodiment, the bacterium is Acidovorax temperans, Citrobacter amalonaticus, Providencia stuartii or Pseudomonas putida.

The present invention relates to method, compounds for use in and/or uses for treating a bacterial infection, where each bacterium may be selected from a gram-negative aerobe organism selected from the following list: Acinetobacter baumannii, Acinetobacter baumanni anitratus, Acinetobacter pittii, Citrobacter freundii complex, Citrobacter koseri, Haemophilus parainfluenzae, Haemophilus paraphrophilus, Klebsiella oxytoca, Klebsiella variicola, Leclercia adecarboxylata, Proteus hauseri; Proteus peneri or Serratia marcescens.

In another aspect, the present invention also provides that each of the gram-negative aerobe organisms and/or gram-positive aerobe organisms as defined above and herein throughout the present application may be non-drug resistant and/or drug-resistant organisms. Reference may be made to any of the organisms being drug-resistant or non-drug resistant (i.e., with an example of this, such as reciting, but not limited to drug-resistant Staphylococcus saprophytrcus or non-drug resistant Staphylococcus saprophyticus or drug-resistant Escherichia coli(E. coli) and drug-resistant K. pneumoniae.

As used herein, the term “drug-resistant” bacterium is synonymous with “non-susceptible” bacterium, and refers to a form of the bacterium which resists the effects of, or has reduced or no susceptibility to, an antibiotic. “Drug-resistant bacterium” includes, for example, bacteria which produce extended-spectrum beta-lactamases (ESBLs); bacteria which produce carbapenemases (such as KPC, GES, OXA-48-like, NDM, VIM and IMP); bacteria which are resistant to carbapenems due to OprD loss or efflux; bacteria which produce AmpC; and bacteria having mutations in the Quinolone Resistance Determining Regions (QRDR) of gyrA and parC genes.

In the present invention, in one embodiment, drug resistance of a bacterium may be suspected (for example through knowledge of the patient's medical history, e.g. recurrent UTI). In another embodiment, drug resistance may be proven through established techniques, which include phenotypic or genotypic determination.

In one embodiment, in the present invention, “drug resistant” means resistance or non-susceptibility as defined by M100 CLSI.

In one embodiment, the bacterium causing the UTI is resistant or suspected to be resistant to an antibiotic selected from the group consisting of: a fluoroquinolone antibiotic including ciprofloxacin and levofloxacin; ampicillin; amoxicillin/clavulanate; trimethoprim-sulfamethoxazole; cefazolin; azithromycin; methicillin; tetracycline; nitroxoline; mecillinam; ceftriaxone, cefixime; nitrofurantoin; and fosfomycin. In one embodiment, the bacterium causing the UTI is multi-drug resistant. In one embodiment, “multi-drug resistant” in the present invention means resistant to two clinically relevant antibiotic classes. In one embodiment, “multi-drug resistant” means resistant to three or more clinically relevant antibiotic classes.

In another aspect, the present invention provides or relates to where gram-negative and/or gram-positive aerobe or anaerobe organism(s) is drug-resistant to antibiotics selected from, but not limited to the group consisting of ciprofloxacin, azithromycin and tetracycline.

In one embodiment, in any of the aspects of the invention, the bacterium is selected from: drug-resistant Klebsiella pneumoniae and drug-resistant Escherichia coli. In one embodiment, the drug-resistant Staphylococcus saprophyticus, drug-resistant Klebsiella pneumoniae, or drug-resistant Escherichia coli are respectively resistant to one or more of an antibiotic selected from: a fluoroquinolone antibiotic including ciprofloxacin and levofloxacin; ampicillin; amoxicillin/clavulanate; trimethoprim-sulfamethoxazole; cefazolin; azithromycin; methicillin; tetracycline; nitroxoline; mecillinam; ceftriaxone, cefixime; nitrofurantoin; and fosfomycin. In one embodiment, the drug-resistant Staphylococcus saprophyticus, drug-resistant Citrobacter koseri and the drug resistant Klebsiella pneumoniae, is respectively resistant to ampicillin.

In another aspect, the present invention relates to a method, compound for use in and/or use for treating a bacterial infection(s) or other diseases as defined in the present invention caused by a gram-negative and/or gram-positive aerobe organism, where the gram-negative aerobe organism, Escherichia coli or K. pneumoniae is drug resistant to antibiotics selected from, but not limited to ampicillin, trimethoprim-sulfamethoxazole and ciprofloxacin/levofloxacin and cefazolin.

In one embodiment, the gram-negative and/or gram-positive aerobe organism is drug-resistant to antibiotics selected from, but not limited to the group selected from ciprofloxacin, azithromycin or tetracycline.

In one embodiment, the drug-resistant bacterium is E. coli which is resistant to two or more classes of antibiotics.

In one embodiment, the drug-resistant bacterium is E. coli which is resistant to three or more classes of antibiotics.

In one embodiment, the drug-resistant organism is:

a) Staphylococcus saprophyticus or Citrobacter koseri;
b) Staphylococcus saprophyticus resistant to ampicillin;
c) Staphylococcus saprophyticus resistant to methicillin;
d) Citrobacter koseri resistant to ampicillin;
e) Klebsiella pneumoniae resistant to ampicillin; or
f) Escherichia coli resistant to ampicillin;
g) Escherichia coli resistant to trimethoprim-sulfamethoxazole;
h) Escherichia coli resistant to ciprofloxacin;
i) Escherichia coli resistant to cefazolin;
j) Escherichia coli resistant to ampicillin, ciprofloxacin and trimethoprim-sulfamethoxazole;
k) Escherichia coli resistant to ampicillin, cefazolin and trimethoprim-sulfamethoxazole;
I) Staphylococcus epidermidis resistant to methicillin.

In one aspect, the present invention provides a method for treating uncomplicated UTI, comprising administering gepotidacin or a pharmaceutically acceptable salt thereof in a therapeutically effective amount in a human in need thereof, wherein the uncomplicated UTI is caused by one or more bacterium selected from: Staphylococcus saprophyticus, drug-resistant Staphylococcus saprophyticus, Proteus hauseri, Proteus peneri, Morganella morganii, Providencia rettgeri, Acidovorax temperans, Citrobacter amalonaticus, Providencia stuartii, Pseudomonas putida, drug-resistant Klebsiella pneumoniae, and drug-resistant Escherichia coli.

In one aspect, the present invention provides a method for treating uncomplicated UTI, comprising administering gepotidacin or a pharmaceutically acceptable salt thereof in a therapeutically effective amount in a human in need thereof, wherein the uncomplicated UTI is caused by one or more bacterium selected from: Staphylococcus saprophyticus and drug-resistant Staphylococcus saprophyticus.

In one aspect, the present invention provides a method for treating uncomplicated UTI, comprising administering gepotidacin or a pharmaceutically acceptable salt thereof in a therapeutically effective amount in a human in need thereof, wherein the uncomplicated UTI is caused by a drug-resistant E. coli.

In one aspect, the present invention provides a method for treating uncomplicated UTI, comprising administering gepotidacin or a pharmaceutically acceptable salt thereof in a therapeutically effective amount in a human in need thereof, wherein the uncomplicated UTI is caused by a multi-drug resistant E. coli.

In one embodiment, in any of the aspects of the invention, the bacterium is selected from: Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans, Staphylococcus warneri, Streptococcus anginosus, Streptococcus australis, Streptococcus constellatus, Streptococcus cristatus, Streptococcus gordonii, Streptococcusinfantarius, Streptococcus infantis, Streptococcus intermedius, Streptococcus massiliensis, Streptococcus mitis, Streptococcus oralis, Streptococcus mutans, Streptococcus parasanguinis, Streptococcus salivarius, Streptococcus sanguinis and Streptococcus vestibularis. In one embodiment, the present invention provides a method for treating a urinary tract infection caused by a gram-positive aerobe organism selected from Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans, Staphylococcus warneri, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massiliensis, Streptococcus mitis, Streptococcus oralis, Streptococcus mutans, Streptococcus parasanguinis, Streptococcus salivarius, Streptococcus sanguinis, and Streptococcus vestibularis, which comprises administering gepotidacin or a pharmaceutically acceptable salt thereof to a subject in need thereof.

In one embodiment, the present invention provides a method for treating a bacterial infection selected from: bloodstream infections, upper respiratory tract infections, lower respiratory tract infections, skin infections, soft tissue infections, intra-abdominal infections, gastro-intestinal infections, genital tract infections and urinary tract infections; caused by a gram-positive aerobe organism selected from Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans, Staphylococcus warneri, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massiliensis, Streptococcus mitts, Streptococcus oralis, Streptococcus mutans, Streptococcus parasanguinis, Streptococcus salivarius, Streptococcus sanguinis, and Streptococcus vestibularis, which comprises administering gepotidacin or a pharmaceutically acceptable salt thereof to a subject in need thereof.

In one embodiment, the gram-positive aerobe organism is selected form a coagulase-negative staphylococci selected from S. capitis, S. caprae, S. cohnii, S. epidermidis, S. haemolyticus, S. hominis, S. intermedius, S. simulans and S. warneri, or a viridans streptococci is selected from S. anginosus, S. australis, S. constellatus, S. cristatus, S. gordonii, S. infantarius, S. infantis, S. intermedius, S. massilliensis, S. mutans, S. oralis, S. parasanguinis, S. salivarius, S. sanguinis, and S. vestibularis.

In one embodiment, in any of the aspects of the invention, the bacterium is selected from: Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans, Staphylococcus warneri, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massilliensis, Streptococcus mitis, Streptococcus oralis, Streptococcus mutans, Streptococcus parasanguinis, Streptococcus salivarius, Streptococcus sanguinis and Streptococcus vestibularis.

In one embodiment, the bacterium is Streptococcus agalactiae, Streptococcus group F, Streptococcus group G, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massilliensis, Streptococcus mitis, Streptococcus oralis, Streptococcus mutans, Streptococcus oralis, Streptococcus parasanguinis, Streptococcus salivarius, Streptococcus sanguinis, or Streptococcus vestibularis.

In one embodiment, the bacterium is Streptococcus agalactiae.

In another embodiment, the bacterium is Staphylococcus lugdenensis, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans or Staphylococcus warneri.

In another aspect, the present invention relates to a method for treating a urinary tract infection caused by a gram-positive aerobe organism selected from: coagulase-negative staphylococci selected from S. capitis, S. caprae, S. cohnii, S. epidermidis, S. haemolyticus, S. hominis, S. intermedius, S. simulans and S. warneri; which comprises administering gepotidacin or a pharmaceutically acceptable salt thereof to a patient in need thereof.

In another aspect, the present invention relates to a method for treating a urinary tract infection caused by a gram-positive aerobe organism selected from: coagulase-negative staphylococci is selected from S. capitis, S. caprae, S. cohnii, S. epidermidis, S, haemolyticus, S. hominis, S. intermedius, S. simulans and S. warneri; which comprises administering a pharmaceutical composition comprising [a] gepotidacin or a pharmaceutically acceptable salt thereof; and [b] at least one or more pharmaceutically acceptable excipient(s) to a patient in need thereof.

In another aspect, the present invention relates to a method for treating a urinary tract infection caused by gram-positive aerobe organism(s) as described herein, where the urinary tract infection is selected from uncomplicated Urinary Tract Infections (uUTI), cystitis and acute cystitis.

In another aspect, the present invention relates to a method for treating cystitis or acute cystitis caused by a gram-positive aerobe organism selected from: coagulase-negative staphylococci is selected from S. capitis, S. caprae, S. cohnii, S. epidermidis, S. haemolyticus, S. hominis, S. intermedius, S. simulans and S. warneri, which comprises administering gepotidacin or a pharmaceutically acceptable salt thereof to a patient in need thereof.

In another aspect, the present invention relates to a method for treating cystitis or acute cystitis caused by a gram-positive aerobe organism selected from: coagulase-negative staphylococci selected from S. capitis, S. caprae, S. cohnii, S. epidermidis, S. haemolyticus, S. hominis, S. intermedius, S. simulans and S. warneri; which comprises administering a pharmaceutical composition comprising [a] gepotidacin or a pharmaceutically acceptable salt thereof; and [b] at least one or more pharmaceutically acceptable excipient(s) to a patient in need thereof.

In another aspect, the present invention relates to a method for treating uncomplicated Urinary Tract Infections (uUTI) caused by a gram-positive aerobe organism selected from: coagulase-negative staphylococci is selected from S. capitis, S, caprae, S, cohnii, S. epidermidis, S. haemolyticus, S. hominis, S. intermedius, S. simulans and S. warneri; which comprises administering gepotidacin or a pharmaceutically acceptable salt thereof to a patient in need thereof.

In another aspect, the present invention relates to a method for treating uncomplicated Urinary Tract Infections (uUTI) caused by a gram-positive aerobe organism which is: coagulase-negative staphylococci selected from S. capitis, S. caprae, S. cohnii, S. epidermidis, S. haemolyticus, S. hominis, S. intermedius, S. simulans and S. warneri; which comprises administering a pharmaceutical composition comprising:

[a] gepotidacin or a pharmaceutically acceptable salt thereof; and

[b] at least one or more pharmaceutically acceptable excipient(s)

to a patient in need thereof.

In another aspect, the present invention relates to methods for treating a urinary tract infection caused by gram-positive aerobe organism(s) as described throughout the instant application, where the gram-positive aerobe organism is non-drug resistant or drug-resistant.

In another aspect, the present invention provides a method for treating a bacterial infection selected from: bloodstream infections, upper respiratory tract infections, lower respiratory tract infections, skin infections, soft tissue infections, intra-abdominal infections, gastro-intestinal infections, genital tract infections and urinary tract infections; caused by a by gram-negative anaerobe organism(s) and/or gram-positive anaerobe organism(s), selected from: Bilophila wadsworthia, Sutterella wadsworthensis, Clostridium bifermentans, Clostridium difficile, Eggethella lenta, Peptostreptococcus anaerobius, Peptostreptococcus anaerobius, Bacteroides caccae, Bacteroides fragilis, Bacteroides ovatus, Bacteroides stercoris, Bacteroides thetaiotaomicron, Bacteroides uniformis; Bacteroides vulgatus; Fusobacterium necrophorum, Fusobacterium nucleatum, Porphyromonas asaccharolytica, Porphyromonas endodontalis, Porphyromonas gingivalis, Porphyromonaslevii, Porphyromonas somerae, Prevotella bivia, Prevotella buccae, Prevotella denticola, Prevotella disiens, Prevotella melaninogenica, Veillonella alcalescens dispar, Veillonella parvula, Bifidobacterium adolescentis, Bifidobacterium breve, Bifidobacterium dentium Bifidobacterium longum, Bifidobacteriumpseudocatenulatum, Collinsella (Eubacterium) aerofaciens, Eubacterium limosum, Eubacterium nodatum, Lactobacillus acidophilus; Lactobacillus crispatus, Lactobacillus fermentum, Lactobacillus gasseri, Lactobacillus iners, Lactobacillus jensenii, Lactobacillus plantarum, and Lactobacillus rhamnosus, which comprises administering gepotidacin or a pharmaceutically acceptable salt thereof to a subject in need thereof.

In another aspect, the present invention relates to a method for treating bacterial infection caused by a gram-negative anaerobe organism(s) and/or gram-positive anaerobe organism(s), as described herein, which comprises administering gepotidacin or a pharmaceutically acceptable salt thereof to a patient in need thereof.

In another aspect, the present invention relates to a method for treating bacterial infection caused by a gram-negative anaerobe organism(s) and/or gram-positive anaerobe organism(s), as described herein, which comprises administering gepotidacin to a patient in need thereof.

In another aspect, the present invention relates to a method for treating bacterial infection caused by a gram-negative anaerobe organism(s) and/or gram-positive anaerobe organism(s), as described herein, which comprises administering a pharmaceutical composition comprising:

[a] gepotidacin or a pharmaceutically acceptable salt thereof; and

[b] a pharmaceutically acceptable excipient(s),

to a patient in need thereof.

In another aspect, the present invention relates to a method, compound for use in and/or use for treating bacterial infection caused by:

Bilophila wadsworthia, Sutterella wadsworthensis, Clostridium bifermentans, Clostridium difficile, Eggethella lenta, Peptostreptococcus anaerobius, Peptostreptococcus anaerobius, Bacteroides caccae, Bacteroides fragilis, Bacteroides ovatus, Bacteroides stercoris, Bacteroides thetaiotaomicron, Bacteroides uniformis, Bacteroides vulgatus Fusobacterium necrophorum, Fusobacterium nucleatum, Porphyromonas asaccharolytica, Porphyromonas endodontalis, Porphyromonas gingivalis, Porphyromonaslevii, Porphyromonas somerae, Prevotella bivia, Prevotella buccae, Prevotella denticola, Prevotella disiens, Prevotella melaninogenica, Veillonella alcalescens dispar, Veillonella parvula, Bifidobacterium adolescentis; Bifidobacterium breve, Bifidobacterium dentium, Bifidobacterium longum, Bifidobacterium pseudocatenulatum, Collinsella (Eubacterium) aerofaciens, Eubacterium limosum, Eubacterium nodatum, Lactobacillus acidophilus, Lactobacillus crispatus, Lactobacillus fermentum, Lactobacillus gasseri, Lactobacillus iners, Lactobacillus jensenii, Lactobacillus plantarum, or Lactobacillus rhamnosus;

which comprises administering gepotidacin or a pharmaceutically acceptable salt thereof to a patient in need thereof.

In one embodiment, the bacterial infection is caused by a Bacteroides species which is resistant to ceftriaxone, clindamycin, imipenem, moxifloxacin or piperacillin-tazobactam, wherein the Bacteroides species is selected from: Bacteroides caccae, Bacteroides fragillis, Bacteroides ovatus, Bacteroides stercoris, Bacteroides thetaiotaomicron, Bacteroides uniformis and Bacteroides vulgatus.

In one embodiment, the bacterial infection is caused by one or more bacterium selected from: Porphyromonas levii which is resistant to metronidazole, Sutterella wadsworthensis which is resistant to metronidazole, Bifidobacterium pseudocatenulatum which is resistant to clindamycin, Clostridioides difficile which is resistant to ceftriaxone, clindamycin, imipenem or moxifloxacin, Eggerthella lenta which is resistant to ceftriaxone, clindamycin or moxifloxacin, Eubacterium nodatum which is resistant to metronidazole, Peptostreptococcus anaerobius which is resistant to clindamycin or moxifloxacin.

In another aspect, the present invention relates to a method for treating a bacterial infection caused by a gram-negative and/or gram-positive anaerobe organism, as described herein, where the a gram-negative and/or gram-positive anaerobe organism is non-drug resistant or drug-resistant.

The invention also provides for methods, compounds for use and/or uses for treating bacterial infections caused by gram-positive and gram-negative aerobes or anaerobes, as described herein, which comprises administering a pharmaceutical composition comprising gepotidacin or a pharmaceutically acceptable salt thereof, and a pharmaceutically acceptable carrier, to a patient in need thereof.

In another aspect the present invention relates to a method, compound for use in and/or use for treating a bacterial infection caused by a gram-negative or gram-positive aerobic or anaerobic organism, where the gram-negative aerobic organism is:

Acinetobacter spp. selected from Acinetobacter baumannii, Acinetobacter baumannii anitratus, and Acinetobacter pittii;

Haemophilus spp. selected from Haemophilus parainfluenzae and Haemophilus paraphrophilus;

Klebsiella spp. selected from Klebsiella oxytoca and Klebsiella variicola; or

Proteus spp. selected from Proteus hauseri and Proteus peneri.

Indications Treated by Methods/Uses

In another aspect of the present invention, the urinary tract infections as defined herein, also may include acute urinary tract infections, where such bacterial infections occur as a result of an abrupt, sudden onset and severity, short duration or rapidly progressive onset and in need of urgent care.

In another aspect, the present invention relates to a method, compound for use in and/or use for treating a bacterial infection(s) caused by a gram-negative or gram-positive aerobe or aerobic or anaerobic organism(s), where the bacterial infection is selected from urinary tract infections in general, which may be, but not limited to an uncomplicated urinary tract infection and/or an acute urinary tract infection, respectively, which may include, but is not limited to cystitis or acute cystitis.

Cystitis is an infection of the bladder. In accordance with the present invention, acute cystitis is a sudden inflammation of the bladder as caused by a bacterial infection, commonly referred to as a urinary tract infection (UTI). See for example “Guidelines on Urological Infections” (European Urological Society), https.//uroweb.org/guideline/urological-infections, which explains “uncomplicated UTI” to be “acute, sporadic or recurrent lower (uncomplicated cystitis) and/or upper (uncomplicated pyelonephritis) UTI, limited to non-pregnant women with no known relevant anatomical and functional abnormalities within the urinary tract or comorbidities.” Symptoms of acute cystitis can come on suddenly, where common symptoms include: frequent and strong urge to urinate even after emptying the bladder; dysuria, a painful or burning sensation when urinating; foul- or strong-smelling urine; cloudy urine; a sensation of pressure, bladder fullness, or cramping in the middle of the lower abdomen or back; a low-grade fever; chills; and/or the presence of blood in the urine.

As used herein, “cystitis” generally refers to an inflammation of the bladder and is the most common type of “UTI.” As used herein, “acute uncomplicated cystitis” is synonymous to “uncomplicated UTI” or “uUTI”.

uUTI is seen in otherwise healthy subjects, mostly female, without relevant structural and functional abnormalities within the urinary tract, kidney diseases or comorbidity that could lead to more serious outcomes and require additional attention.

As used herein, “recurrent uUTI” means recurrences of uncomplicated UTI, with a frequency of at least three episodes a year or two episodes in the last six months. Treatment of recurrent uUTI requires special considerations such as diagnosis of infection by urine culture, for example identification of a uropathogen in a mid stream urine sample. For example, in one embodiment, a bacterial count of at least 10³ CFU/mL indicates recurrent uUTI.

UTI can also be “complicated UTI”, which is an infection associated with a condition, such as a structural or functional abnormality of the genitourinary tract, or the presence of an underlying disease, which increase the risk of a more serious outcome than expected from UTI in individuals without an identified risk factor or of failing therapy. In the present invention, “treatment of UTI” includes treatment of uncomplicated UTI and treatment of complicated UTI.

In one embodiment, in any aspect of the present invention, the urinary tract infection is selected from uncomplicated Urinary Tract Infections (uUTI), cystitis and acute cystitis.

In one embodiment, in any aspect of the present invention, the UTI is uncomplicated UTI.

In one embodiment, in any aspect of the present invention, the UTI is complicated UTI.

In one embodiment, in any aspect of the present invention, the human is female.

In one embodiment, in any aspect of the present invention, the gepotidacin is gepotidacin free base.

In one embodiment, in any aspect of the present invention, the gepotidacin is gepotidacin methanesulphonate.

In one embodiment, in any aspect of the present invention, the UTI is recurrent uncomplicated UTI.

In one embodiment, in any aspect of the present invention, the human is pregnant, adolescent or paediatric. As used herein, “adolescent” means aged 12, 13, 14, 15, 16 or 17 year old (i.e. age 12-17 inclusive). As used herein, “paediatric” means aged 11 or under.

In one embodiment, in any aspect of the present invention, the human has failed at least one prior line of treatment. In one embodiment, the prior line of treatment may be an antibiotic such as a cephalosporin, carbapenems, nitrofurantoin, trimethoprim alone or combined with a sulphonamide, amoxicillin-clavulanate, fosfomycin or a fluoroquinolone such as ciprofloxacin, levofloxacin, gemifloxacin, moxifloxacin, norfloxacin or ofloxacin. Failure of treatment may be defined according to established guidelines; for example, lack of improvement of symptoms after a 3, 4, 5, 6, or 7 day treatment with an antibiotic may be considered a failure.

In another aspect, the present invention relates to a method, compound for use in and/or use for treating a bacterial infection(s) caused by Staphylococcus saprophyticus, where the pharmaceutically acceptable salt of gepotidacin is an acid addition salt.

In another aspect, the present invention relates to a method, compound for use in and/or use for treating a bacterial infection(s) caused by Staphylococcus saprophyticus, where the gepotidacin acid addition salt is formed from: [a] mineral acids selected from hydrochloric acid, hydrobromic acid, sulphuric acid, nitric acid and phosphoric acid; or [b] organic acids selected from acetic acid, fumaric acid, succinic acid, maleic acid, citric acid, benzoic acid, p-toluenesulphonic acid, methanesulphonic acid, naphthalenesulphonic acid and tartaric acid.

In another aspect, the present invention relates to a method, compound for use in and/or use for treating a bacterial infection(s) caused by Staphylococcus saprophyticus, wherein the gepotidacin acid addition salt is a methane sulphonic acid salt.

In another aspect, the present invention relates to a method, compound for use in and/or use for treating a bacterial infection(s) caused by Staphylococcus saprophyticus, which comprises administering gepotidacin or a pharmaceutically acceptable salt thereof to a patient in need thereof.

In another aspect, the present invention relates to a method, compound for use in and/or use for treating a bacterial infection(s) caused by Staphylococcus saprophyticus infection, which comprises administering a pharmaceutical composition comprising:

[a] gepotidacin or a pharmaceutically acceptable salt thereof; and

[b] a pharmaceutically acceptable excipient(s)

to a patient in need thereof.

In another aspect, the present invention relates to a method, compound for use in and/or use for treating a urinary tract infection caused by Staphylococcus saprophyticus, which comprises administering gepotidacin or a pharmaceutically acceptable salt thereof to a patient in need thereof.

In another aspect, the present invention relates to a method for treating a urinary tract infection caused by Staphylococcus saprophyticus and/or a gram negative aerobe, which comprises administering gepotidacin or a pharmaceutically acceptable salt thereof to a patient in need thereof; wherein the gram-negative aerobe organism is selected from: Acinetobacter baumannii anitratus, Acinetobacter pittii; Haemophilus parainfluenzae; Haemophilus paraphrophilus; Klebsiella oxytoca or Klebsiella variicola; Proteus hauseri or Proteus peneri.

In another aspect, the present invention relates to a method, compound for use in and/or use for treating a urinary tract infection caused by Staphylococcus saprophyticus, drug-resistant E. coli, or drug-resistant K. pneumoniae, which comprises administering gepotidacin or a pharmaceutically acceptable salt thereof to a patient in need thereof.

In another aspect, the present invention relates to a method, compound for use in and/or use for treating cystitis caused by Staphylococcus saprophyticus, drug-resistant E. coli, or drug-resistant K. pneumoniae which comprises administering gepotidacin or a pharmaceutically acceptable salt thereof to a patient in need thereof.

In another aspect, the present invention relates to a method, compound for use in and/or use for treating acute cystitis caused by Staphylococcus saprophyticus, drug-resistant E. coli, or drug-resistant K. pneumoniae, which comprises administering gepotidacin or a pharmaceutically acceptable salt thereof to a patient in need thereof.

In another aspect, the present invention as described herein relates to a method, compound for use in and/or use for treating bacterial infections as caused by organism defined in the present application and more specific infections, which may include, but are not limited to Urinary Tract Infection, which includes, but is not limited to uncomplicated Urinary Tract Infection (uUTI) and/or cystitis, which may include, but is not limited to acute cystitis as defined herein.

In another aspect, the present invention also relates to:
a method, compound for use in and/or use for treating a bacterial infection(s) caused by a gram-positive aerobe and/or gram-negative aerobe;
a method, compound for use in and/or use for treating a bacterial infection(s) caused by a gram-positive aerobe organism, such as, but not limited to Staphylococcus saprophyticus;
a method, compound for use in and/or use for treating urinary tract infections, which include, but is not limited to:
   uncomplicated Urinary Tract Infections (uUTI);
   cystitis;
   acute cystitis;
   complicated UTI;
a method, compound for use in and/or use for treating a bacterial infection(s) such as uncomplicated Urinary Tract Infection (uUTI) and/or cystitis, which may include, but is not limited to acute cystitis, caused by drug-resistant E. coli or drug-resistant K. pneumoniae,
a method, compound for use in and/or use for treating a bacterial infection(s) uncomplicated Urinary Tract Infection (uUTI) and/or cystitis, which may include, but is not limited to acute cystitis, caused by S. saprophyticus;
a method, compound for use in and/or use for treating a bacterial infection(s) uncomplicated Urinary Tract Infection (uUTI) and/or cystitis, which may include, but is not limited to acute cystitis, caused by gram-negative aerobes;
where:

each of the aforementioned or above-identified methods or uses, respectively comprise administration of gepotidacin or pharmaceutically acceptable salts thereof and/or corresponding pharmaceutical compositions thereof, respectively, as defined herein throughout the instant specification.

In another aspect, the present invention relates to methods, compounds for use in and/or uses for treating cystitis caused by Staphylococcus saprophyticus, where the cystitis is acute cystitis.

In another aspect, the present invention relates to use of a compound as defined herein and throughout the instant specification, where the cystitis caused by Staphylococcus saprophyticus, drug-resistant E. coli; or drug-resistant K. pneumoniae is an acute cystitis.

In one embodiment, the present invention provides a method for treating cystitis caused by Staphylococcus saprophyticus, which comprises administering gepotidacin or a pharmaceutically acceptable salt thereof to a patient in need thereof.

In one embodiment, the present invention provides a method for treating uncomplicated urinary tract infections, cystitis or acute cystitis, which comprises administering gepotidacin or a pharmaceutically acceptable salt thereof to a patient in need thereof.

In one embodiment, the present invention provides a method for treating uncomplicated urinary tract infections, cystitis or acute cystitis, which comprises administering gepotidacin to a patient in need thereof.

In one embodiment, the present invention provides a method for treating uncomplicated urinary tract infections, cystitis or acute cystitis, which comprises administering a pharmaceutical composition comprising:

[a] gepotidacin or a pharmaceutically acceptable salt thereof; and

[b] a pharmaceutically acceptable excipient(s)

to a patient in need thereof.

In one embodiment, the present invention provides a method for treating a urinary tract infection caused by a gram-negative aerobe which comprises administering gepotidacin or a pharmaceutically acceptable salt thereof to a patient in need thereof.

In one embodiment, the present invention provides gepotidacin or a pharmaceutically acceptable salt thereof for the treatment of uncomplicated urinary tract infections, cystitis or acute cystitis, caused by Staphylococcus saprophyticus, drug-resistant E. coli, or drug-resistant K. pneumoniae.

In one embodiment, the present invention provides use of gepotidacin or a pharmaceutically acceptable salt thereof for the treatment of uncomplicated urinary tract infections, cystitis or acute cystitis, caused by Staphylococcus saprophyticus, drug-resistant E. coli, drug-resistant K. pneumoniae or a gram-negative aerobe.

In another aspect, the present invention relates to use of gepotidacin or a pharmaceutically acceptable salt thereof for the treatment of urinary tract infection caused by Staphylococcus saprophyticus.

In another aspect, the present invention relates to use of gepotidacin or a pharmaceutically acceptable salt thereof for the treatment of cystitis caused by Staphylococcus saprophyticus.

In another aspect, the present invention relates to use of gepotidacin or a pharmaceutically acceptable salt thereof for the treatment of acute cystitis caused by Staphylococcus saprophyticus.

In another aspect, the present invention relates to use of gepotidacin or a pharmaceutically acceptable salt thereof for the treatment of urinary tract infections caused by gram-negative aerobes.

In another aspect, the present invention relates to methods, compounds for use in and/or uses for treating infection caused by:
a gram-positive organism selected from Staphylococcus saprophyticus; or
a gram-negative aerobe organism selected from Acinetobacter baumannii, Acinetobacter baumannii anitratus, Acinetobacter pittii; Citrobacter freundii complex, Citrobacter koseri, Haemophilus parainfluenzae, Haemophilus paraphrophilus, Klebsiella oxytoca, Klebsiella variicola, Leclercia adecarboxylata, Proteus hauseri Proteus peneri and Serratia marcescens;

which comprises administering:

[a] a pharmaceutical composition comprising gepotidacin or a pharmaceutically acceptable salt thereof; and

[b] a pharmaceutically acceptable excipient(s);

to a subject in need thereof.

In another aspect, the present invention relates to methods, compounds for use in and/or uses for treating uncomplicated Urinary Tract Infection (uUTI), cystitis or acute cystitis caused by drug-resistant E. coli or drug-resistant K. pneumoniae, which comprises administering gepotidacin or a pharmaceutically acceptable salt thereof to a patient in need thereof.

In another aspect, the present invention relates to methods, compounds for use in and/or uses for treating uncomplicated Urinary Tract Infection (uUTI), cystitis or acute cystitis caused by drug-resistant E. coli or drug-resistant K. pneumoniae, which comprises administering gepotidacin to a patient in need thereof.

In another aspect, the present invention relates to methods, compounds for use in and/or uses for treating uncomplicated Urinary Tract Infection (uUTI), cystitis or acute cystitis caused by drug-resistant E. coli or drug-resistant K. pneumoniae, which comprises administering a pharmaceutical composition comprising:

[a] gepotidacin or a pharmaceutically acceptable salt thereof; and

[b] pharmaceutically acceptable excipient(s)

to a patient in need thereof.

In another aspect, the present invention relates to methods, compounds for use in and/or uses for treating cystitis caused by Staphylococcus saprophyticus, drug-resistant E. coli, or drug-resistant K. pneumoniae which comprises administering gepotidacin or a pharmaceutically acceptable salt thereof to a patient in need thereof.

In another aspect, the present invention relates to a method, compound for use in and/or use for treating a bacterial infection(s) or other diseases as defined in the present application caused by a gram-negative and/or gram-positive aerobe, where a gram-negative and/or gram-positive aerobe organism is non-drug resistant or drug-resistant.

In another aspect, the present invention relates to a method, compound for use in and/or use for treating a bacterial infection(s) or other disease(s) as defined in the present application caused by a gram-negative and/or gram-positive aerobe organism, where the gram-negative and/or gram-positive aerobe organism is drug-resistant to antibiotics and is selected from, but not limited to the group selected from fluoroquinolones (including ciprofloxacin), azithromycin, tetracycline, nitrofurantoin, trimethoprim/sulfamethoxazole and fosfomycin.

In another aspect, the present invention relates to a method, compound for use in and/or use for treating a bacterial infection(s) or other disease(s) as defined in the present application caused by a gram-negative and/or gram-positive aerobe organism, where the gram-negative aerobe organism, drug-resistant Escherichia coli or drug-resistant K. pneumoniae is resistant to antibiotics selected from, but not limited to ampicillin, trimethoprim-sulfamethoxazole and ciprofloxacin/levofloxacin or cefazolin.

In another aspect, the present invention relates to gepotidacin or a pharmaceutically acceptable salt thereof for use in treating uncomplicated Urinary Tract Infection (uUTI), cystitis or acute cystitis caused by drug-resistant E. coli or drug-resistant K. pneumoniae in a patient in need thereof.

In another aspect, the present invention relates to use of a pharmaceutical composition comprising:

[a] gepotidacin or a pharmaceutically acceptable salt thereof; and

[b] a pharmaceutically acceptable excipient(s)

in treating uncomplicated Urinary Tract Infection (uUTI), cystitis or acute cystitis caused by drug-resistant E. coli or drug-resistant K. pneumoniae in a patient in need thereof.

In another aspect, the present invention relates to gepotidacin or a pharmaceutically acceptable salt thereof for use in treating cystitis caused by Staphylococcus saprophyticus, drug-resistant Escherichia coli, or drug-resistant Klebsiella pneumoniae in a patient in need thereof.

In another aspect, the present invention relates to a method, compound for use in and/or use for treating urinary tract infections caused by:
a gram-positive organism selected from Staphylococcus saprophyticus, or
a gram-negative aerobe organism selected from Acinetobacter spp., Citrobacter spp., Haemophilus spp., Klebsiella spp., Leclercia adecarboxylata, Proteuss spp. and Serratia marcescens; wherein
Acinetobacter spp. is selected from Acinetobacter baumannii, Acinetobacter anitratus, and Acinetobacter pittii;
Citrobacter spp. is selected from Citrobacter freundii complex and Citrobacter koseri,
Haemophilus spp. is selected from H. parainfluenzae and H. paraphrophilus;
Klebsiella spp. is selected from Klebsiella oxytoca and Klebsiella variicola, or
Proteus spp. is selected from Proteus hauseri and Proteus peneri

which comprises administering gepotidacin or a pharmaceutically acceptable salt thereof to a patient in need thereof.

In another aspect, the present invention relates to a method, compound for use in and/or use for treating urinary tract infections caused by:
a gram-positive organism selected from Staphylococcus saprophyticus, or
a gram-negative aerobe organism is selected from Acinetobacter spp., Citrobacter spp., Haemophilus spp., Klebsiella spp., Leclercia adecarboxylata, Proteus spp. or Serratia marcescens,
wherein Acinetobacter spp. Is selected from Acinetobacter baumannii, Acinetobacter anitratus, and Acinetobacter pittii;
Citrobacter spp. is selected from Citrobacter freundii complex and Citrobacter koseri,
Haemophilus spp. is selected from H. Parainfluenzae and H. paraphrophilus;
Klebsiella spp. is selected from Klebsiella oxytoca and Klebsiella variicola, and
Proteus spp. is selected from Proteus hauseri and Proteus peneri;

which comprises administering a pharmaceutical composition comprising

[a] gepotidacin or a pharmaceutically acceptable salt thereof; and

[b] a pharmaceutically acceptable excipient(s);

to a patient in need thereof.

In one aspect, the present invention provides for method(s), compound(s) for use in and/or use(s), individually or respectively, as defined herein for treating a bacterial infection, such as UTI, caused by gram-positive aerobe organism(s) where:
the gram-positive aerobe organism is selected from Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans or Staphylococcus warneri, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massilliensis, Streptococcus mitis, Streptococcus oralis, Streptococcus mutans, Streptococcus oralis, Streptococcus parasanguinis, Streptococcus salivarius, Streptococcus sanguinis, and Streptococcus vestibularis.

Compounds Used In The Present Invention

WO2008/128942 discloses the preparation of the free base and the hydrochloride salt of gepotidacin.

Furthermore, it will be understood that the phrase “gepotidacin or a pharmaceutically acceptable salt thereof” is intended to encompass gepotidacin, a pharmaceutically acceptable salt of gepotidacin, a solvate of gepotidacin, or any pharmaceutically acceptable combination of these. Thus by way of non-limiting example used here for illustrative purpose, “gepotidacin or a pharmaceutically acceptable salt thereof” may include a pharmaceutically acceptable salt of gepotidacin that is further present as a solvate.

As used herein, the term “compound(s) of the invention” means a gepotidacin in any form, i.e., any salt or non-salt form (e.g., as a free base, or as a pharmaceutically acceptable salt thereof) and any physical form thereof, e.g., including non-solid forms (e.g., liquid or semi-solid forms), and solid forms (e.g., amorphous or crystalline forms, specific polymorphic forms, solvates, including hydrates), and mixtures of various forms.

Suitable pharmaceutically acceptable salts include those described by Berge, Bighley and Monkhouse J. Pharm. Sci (1977) 66, pp 1-19.

The compound of the invention is a base (contains a basic moiety), and therefore a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenyl propionates, phenylbutrates, citrates, lactates, γ-hydroxybutyrates, glycollates, tartrates mandelates, and sulfonates, such as xylenesulfonates, methanesulfonates, propanesulfonates, naphthalene-1-sulfonates and naphthalene-2-sulfonates.

Pharmaceutically acceptable salts of gepotidacin include the acid addition salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids.

The present invention includes within its scope all possible stoichiometric and non-stoichiometric salt forms.

The invention also includes various deuterated forms of the compounds of the invention or a pharmaceutically acceptable salt thereof. Each available hydrogen atom attached to a carbon atom may be independently replaced with a deuterium atom. For example, deuterated materials, such as alkyl groups may be prepared by conventional techniques (see for example: methyl-d3-amine available from Aldrich Chemical Co., Milwaukee, Wis., Cat. No. 489,689-2).

Pharmaceutical Compositions And Formulations

Pharmaceutical Compositions and Formulations acceptable and adaptable for use in methods and/or uses of the present invention are prepared using conventional art known pharmaceutical compositions, formulation or chemical materials, formulary excipients, preparation means, processes and/or methods and conventional techniques, etc.

In particular, gepotidacin or pharmaceutically acceptable salts thereof, used in the present invention may be formulated for administration in any convenient way for use in human or veterinary medicine, by analogy with other antibacterials/antitubercular compounds.

The pharmaceutical compositions used in the present invention may be formulated for administration by any route and include those in a form adapted for oral, topical or parenteral use and may be used in mammals including humans.

The compositions may be in the form of tablets, capsules, powders, granules, lozenges, suppositories, creams or liquid preparations, such as oral or sterile parenteral solutions or suspensions.

In one embodiment, the gepotidacin or pharmaceutically acceptable salt thereof of the present invention is in a tablet or a capsule form. In one embodiment, it is in a tablet form. In one embodiment, the tablet is a 750 mg tablet.

The formulations may also contain compatible conventional carriers, such as cream or ointment bases and ethanol or oleyl alcohol for lotions.

Tablets and capsules for oral administration in the present invention may be in unit dose presentation form, and may contain conventional excipients such as binding agents, fillers, tabletting lubricants, disintegrants, or wetting agents. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

Moreover, the quantity of the compound or pharmaceutical composition used in the present invention administered will vary depending on the patient and the mode of administration and can be any effective amount.

In accordance with any of the methods of administration of the present invention, the term a “therapeutically effective amount”, as used herein, generally includes within its meaning a non toxic but sufficient amount of the particular drug to which it is referring to provide the desired therapeutic effect. The exact amount required will vary from subject to subject depending on factors such as the patient's general health, the patient's age, etc.

Treatment regimen for the administration of the compounds and/or pharmaceutical compositions used in the present invention can also be determined readily by those with ordinary skill in art. The quantity of the compound and/or pharmaceutical composition used in the present invention administered may vary over a wide range to provide in a unit dosage an effective amount based upon the body weight of the patient per day to achieve the desired effect.

The compositions may contain from 0.1% by weight, preferably from 10-60% by weight, of the active material, depending on the method of administration. Where the compositions comprise dosage units, each unit will preferably contain from 50-1000 mg of the active ingredient. Unless otherwise noted, the amount of the active ingredient (i.e., gepotidacin) refers to that of gepotidacin free base.

The dosage as employed for adult human treatment in the present invention will preferably range from 100 to 3000 mg per day, for instance 1500 mg per day depending on the route and frequency of administration. Such a dosage corresponds to about 1.5 to about 50 mg/kg (mg of gepotidacin per kg of patient body weight) per day. Suitably the dosage is from 5 to 30 mg/kg per day. In one embodiment, the dosage is 1500 mg twice a day (i.e. 3000 mg per day). In one embodiment, the two doses in one day are given 6-12 hours apart.

Thus in one embodiment, the present invention provides a method for treating urinary tract infection (UTI), comprising administering gepotidacin or a pharmaceutically acceptable salt thereof, in a therapeutically effective amount in a human in need thereof, wherein the UTI is caused by one or more bacterium as defined in the first aspect of the invention, and the gepotidacin or a pharmaceutically acceptable salt thereof is administered at 1500 mg twice a day, 6-12 hours apart.

In particular, a composition of the present invention is presented as a unit dose and taken preferably from 1 to 5 times daily, such as once or twice daily to achieve the desired effect. In one embodiment, gepotidacin or its pharmaceutically acceptable salt thereof is administered for any of 3, 4, 5, 6 or 7 continuous days. In one embodiment, in any aspect of the present invention, gepotidacin or its pharmaceutically acceptable salt thereof is administered for 5 continuous days.

Conventional administration methods may be suitable for use in the present invention.

Depending upon the treatment being effected, the compounds, and/or or compositions of the present invention can be administered orally, intravascularly, intraperitoneally, subcutaneously, intramuscularly or topically. Preferably, the composition is adapted for oral administration.

Gepotidacin or a pharmaceutically acceptable salt thereof used in the present invention may be the sole therapeutic agent in the compositions of the invention or a combination with other antibacterials. If the other antibacterial is a β-lactam then a β-lactamase inhibitor may also be employed.

The Examples set forth below are illustrative of the present invention and are not intended to limit, in any way, the scope of the present invention.

### EXAMPLES

Unless otherwise stated, the Clinical and Laboratory Standards Institute (CLSI) recommended procedure is as set out in the edition of CLSI Approved Standard M07. Methods for Dilution Antimicrobial Susceptibility Tests for Bacteria That Grow Aerobically” at the time of testing.

### Example 1

Biological Activity Assay (In Vitro Assays)

Studies were conducted to assess the in vitro activity of compounds of the present invention and the specific comparator compounds as identified in Methods below.

Method 1

Antimicrobial activity was determined by broth microdilution using the CLSI recommended procedure.

Gepotidacin was tested in serial two-fold dilutions and the minimum inhibitory concentration (MIC) was determined as the lowest concentration of the compound that inhibited visible growth.

Gepotidacin was tested against 28 strains of Staphylococcus saprophyticus collected from multiple hospitals.

Amoxicillin, azithromycin, levofloxacin and cefuroxime were included as comparators. MICs for gepotidacin and comparators were determined by broth microdilution according to CLSI methods.

The MIC90 (MIC which inhibits 90% of the isolates tested) for gepotidacin was 0.125 μg/mL against 28 isolates of S. saprophyticus. This MIC90 value was at least 2 to >512-fold lower than the tested comparators.

In addition, gepotidacin and at least one comparator from the list above were evaluated against gram-negative aerobic organisms selected from Acinetobacter baumannii, Acinetobacter baumannii anitratus, Acinetobacter pittii, Citrobacter freundii complex, Citrobacter koseri, Haemophilus parainfluenzae, Haemophilus paraphrophilus, Klebsiella oxytoca, Klebsiella variicola, Leclercia adecarboxylata, and Serratia marcescens.

At least one salt of gepotidacin was tested (i.e., such as mesylate salt) and found to have a MIC of ≤8 μg/mL against at least one strain of every organism listed above.

Method 2

Antimicrobial activity was determined in a second study by agar microdilution using the CLSI recommended procedure in serial two-fold dilutions and the minimum inhibitory concentration (MIC) was determined as the lowest concentration of the compound that inhibited visible growth.

This study tested gepotidacin by CLSI agar dilution against 51 isolates of S. saprophyticus collected between 2005-2018. The MIC90 of the compound against all isolates tested was 0.125 μg/mL.

Amikacin, ceftazidime, colistin, fosfomycin, levofloxacin, meropenem, nitrofurantoin and trimethoprim-sulfamethoxazole were included as comparators. MICs for and comparators were determined by agar dilution according CLSI methods.

In addition, gepotidacin and all of the aforementioned comparators identified in this method were evaluated against gram-negative aerobic organisms selected from Proteus hauseri and Proteus peneri. Gepotidacin was tested in at least one exemplified salt.

Gepotidacin had a MIC of ≤8 μg/mL against at least one strain of every organism listed above.

Conclusion to Methods 1 and 2

These studies demonstrate the in vitro activity of (gepotidacin against Staphylococcus saprophyticus(MIC90=0.12 μg/mL) and also Acinetobacter baumannii, Acinetobacter baumannii anitratus, Acinetobacter pittii, Citrobacter freundii complex, Citrobacter koseri, Haemophilus parainfluenzae, Haemophilus paraphrophilus, Klebsiella oxytoca, Klebsiella variicola, Leclercia adecarboxylata, Proteus hauseri, Proteus peneri and Serratia marcescens (MIC≤ 8 μg/mL against at least one strain of every organism listed).

Additional Information for Method 2

Materials/Methods:

The panel comprised 511 Enterobacterales (previously known as Enterobacteriaceae), including E. coli, Klebsiella oxytoca, Klebsiella variicola, Klebsiella pneumoniae, Enterobacter aerogenes, Enterobacter cloacae complex, Proteus mirabilis, Proteus penneri, Proteus hauseri, and Shigella boydii, Shigella flexneri, and Shigella sonnei enriched to include isolates with ESBLs, AmpC or carbapenemases; 55 Pseudomonas aeruginosa selected to include isolates with carbapenemases, ESBLs, or carbapenem resistance due to OprD loss and/or efflux; 60 Acinetobacter baumannii with carbapenemases; 95 Neisseria gonorrhoeae including those with resistance or reduced susceptibility to beta-lactams, ciprofloxacin, azithromycin, tetracycline or spectinomycin; and 51 Staphylococcus saprophyticus isolates.

MICs were determined according to CLSI agar dilution guidelines.

Results:

Gepotidacin MICs for E. coli ranged≤0.06-64 mg/L, with 96.1% inhibited of the tested isolates at concentrations of ≤8 mg/L. The activity of gepotidacin against E. coil was unaffected by levofloxacin non-susceptibility, or by amino acid substitutions in the QRDR of GyrA and ParC associated with quinolone resistance (available for 188 out of the 254 E. coli isolates). Gepotidacin MIC distribution was also unrelated to beta-lactamase resistance mechanism; 100% of NDM, 94.6% of KPC and 92.1% of OXA-48-like producing E. coli were inhibited at ≤ 8 mg/L gepotidacin.

Gepotidacin MICs for Shigella ranged from 0.125-8 mg/L, with 95.6% of the tested isolates inhibited at ≤ 2 mg/L and 100% inhibited at ≤8 mg/L. Specifically, gepotidacin MICs against Shigella boydii (n=3) was 2 mg/L; gepotidacin MIC90 against Shigella flexneri (n=24) was 2 mg/L and MIC90 against Shigella sonnei (n=64) was 2 mg/L. The activity of gepotidacin was unaffected by levofloxacin non-susceptibility, or by amino acid substitutions in the QRDR of GyrA and ParC (where data was available), and gepotidacin MIC distribution was also unrelated to beta-lactamase resistance mechanism.

Gepotidacin MICs ranged higher for Klebsiella (Klebsiella oxytoca, Klebsiella variicola, Klebsiella pneumoniae), Enterobacter (Enterobacter aerogenes, Enterobacter cloacae complex) and Proteus (Proteus mirabilis, Proteus penneri, Proteus hauseri), with MIC90s of 64, 32, and 32 mg/L, respectively. Specifically, gepotidacin MIC against Klebsiella oxytoca, Klebsiella variicola, Proteus hauseri and Proteus peneri was at least 4 mg/L; in each case against at least one isolate.

MICs for all Enterobacteriaceae tested were unrelated to levofloxacin resistance or to amino acid substitutions in the QRDRs of GyrA and ParC.

MIC90 for P. aeruginosa and A. baumannii were 32 and 64 mg/L respectively, and were unrelated to levofloxacin resistance.

MICs for all S. saprophyticus were ≤0.125 mg/L (same data as shown in Method 2).

TABLE 1

| | Number of Isolates/Gepotidacin MIC (mg/L) | | | | | | | | |
|---|---|---|---|---|---|---|---|---|---|
| Organism (n) | ≤0.25 | 0.5 | 1 | 2 | 4 | 8 | ≥16 | MIC50 | MIC90 |
| E. coli (n = 254) | 4 | 22 | 56 | 92 | 37 | 33 | 10 | 2 | 8 |
| Shigella (n = 91) | 14 | 18 | 36 | 19 | 3 | 1 | | 1 | 2 |
| Klebsiella (n = 55) | | | 1 | | 6 | 14 | 34 | 16 | 64 |
| Enterobacter | 1 | | | 3 | 12 | 7 | 32 | 16 | 32 |
| (n = 55) | | | | | | | | | |
| Proteus (n = 56) | | 3 | 4 | 11 | 9 | 10 | 19 | 8 | 32 |
| P. aeruginosa | | 1 | 2 | 4 | 18 | 15 | 15 | 8 | 32 |
| (n = 55) | | | | | | | | | |
| A. baumannii | | | | | | 7 | 53 | 32 | 64 |
| (n = 60) | | | | | | | | | |
| N. gonorrhoeae | 79 | 6 | 6 | 4 | | | | 0.25 | 1 |
| (n = 95) | | | | | | | | | |
| S. saprophyticus | 51 | | | | | | | ≤0.06 | 0.125 |
| (n = 51) | | | | | | | | | |

Conclusions:

Gepotidacin was active in-vitro against S. saprophyticus (100%≤0.125 mg/L).

At 8 mg/L, gepotidacin was active against drug resistant E. coli (96.1%) and Shigella (100%), but less active against other gram-negative genera tested.

Method 3

Antimicrobial activity was determined by broth microdilution using the CLSI recommended procedure.

The compounds were tested in serial two-fold dilutions and the minimum inhibitory concentration (MIC) was determined as the lowest concentration of the compound that inhibited visible growth.

In particular, gepotidacin was tested against 6 strains of Staphylococcus saprophyticus from the collection of isolates at Laboratory Specialists, Inc., Westlake, Ohio.

Levofloxacin was included as the comparator in the study to determine the effect of urine on the in vitro activity of gepotidacin against Staphylococcus saprophyticus. MICs were determined by broth microdilution according CLSI methods.

The minimum inhibitory concentration (MIC) range for gepotidacin was 0.03-0.5 μg/mL against 3 isolates of methicillin-susceptible S. saprophyticus and 0.06-0.5 μg/mL against 3 isolates of methicillin-resistant S. saprophyticus. The MIC values for gepotidacin were 8-32-fold lower than levofloxacin for the methicillin-susceptible S. saprophyticus and were 4-fold lower than levofloxacin for each of the methicillin-resistant S. saprophyticus.

At least one exemplified salt of gepotidacin was tested (such as mesylate salt).

Gepotidacin had a MIC of ≤0.5 μg/mL against at least one strain of methicillin-susceptible or methicillin-resistant S. saprophyticus.

The data shown in the table below are results from an in vitro study conducted with the same S. saprophyticus isolates described above, to determine the effect of urine on the in vitro activity of gepotidacin and levofloxacin against S. saprophyticus. Study strains were tested for MICs (minimum inhibitory concentrations) according to reference CLSI broth microdilution method using cation-adjusted Mueller Hinton Broth (CAMHB) and with the addition of 25%, 50% and 100% urine (not pH adjusted, pH 6.42) and 100% urine (pH adjusted, 7.31 and 8.07). MIC results (mean dilution difference) were slightly higher (mean dilution differences of 0.67-1.54) for both gepotidacin and levofloxacin in 100% pooled urine but do not appear to be a function of pH.

TABLE 2
Comparison of the CLSI MIC Broth Microdilution Reference Method
(CAMHB) To Urine Conditions

| | | | | | |
|---|---|---|---|---|---|
| | | | Mean | | |
| Comparative | Mean | Mean | Dilution | N(%) ± | N(%) ± |
| Condition | MIC | difference | Difference | 1 dilution | 2 dilution |

Staphylococcus saprophyticus (n = 6)

| | | | | | |
|---|---|---|---|---|---|
| Gepotidacin | | | | | |
| CAMHB (ref. | 0.11 | | | | |
| method, pH 7.2) | | | | | |
| 25% Urine (pH | 0.11 | 0.00 | 0.00 | 6 (100%) | 6 (100%) |
| 7.09) | | | | | |
| 50% Urine (6.99) | 0.11 | 0.00 | 0.00 | 6 (100%) | 6 (100%) |
| 100% Urine (pH | 0.24 | 0.14 | 1.18 | 4 (66.7%) | 6 (100%) |
| 6.42) | | | | | |
| 100% Urine (pH | 0.31 | 0.21 | 1.54 | 3 (50%) | 4 (66.7%) |
| 7.31) | | | | | |
| 100% Urine (pH | 0.28 | 0.17 | 1.36 | 3 (50%) | 5 (83.3%) |
| 8.07) | | | | | |
| Levofloxacin | | | | | |
| CAMHB (ref. | 0.79 | | | | |
| method, pH 7.2) | | | | | |
| 25% Urine (pH | 1.12 | 0.33 | 0.50 | 6 (100%) | 6 (100%) |
| 7.09) | | | | | |
| 50% Urine (6.99) | 0.89 | 0.10 | 0.17 | 6 (100%) | 6 (100%) |
| 100% Urine (pH | 1.26 | 0.47 | 0.67 | 6 (100%) | 6 (100%) |
| 6.42) | | | | | |
| 100% Urine (pH | 2.24 | 1.45 | 1.50 | 2 (33.3%) | 6 (100%) |
| 7.31) | | | | | |
| 100% Urine (pH | 1.78 | 0.99 | 1.17 | 5 (83.3%) | 6 (100%) |
| 8.07) | | | | | |

In conclusion, these studies demonstrate the in vitro activity of gepotidacin against the methicillin-susceptible and methicillin-resistant S. saprophyticus isolates tested with MICs ≤40.5 ug/mL, and slightly higher MICs (mean dilution differences of 1.18-1.54) in 100% urine. That gepotidacin activity is not significantly affected by urine is surprising and makes it a suitable treatment option for UTI.

### Example 2: Distribution and Exposure of Gepotidacin in Tissues and Body Fluids of Healthy and Infected Participants

Antibiotic target site exposures play a key role as suboptimal levels may lead to therapeutic failure and resistance. Across multiple clinical studies, exposures to gepotidacin were assessed in plasma and other matrices, such as urine, saliva, epithelial lining fluid (ELF) and Alveolar Macrophages (AM).

Bronchoalveolar lavage samples were collected to determine gepotidacin exposures in ELF and AM following a single 1000 mg IV infusion over 2 hours to healthy subjects. Saliva exposure was assessed in healthy volunteers following 1500 mg oral single dose. Gepotidacin exposures in urine was assessed in women with acute uUTI following 1500 mg BID oral doses for 5 days.

ELF and AM AUC(0-12) ratios to unbound plasma were 1.84 and 178, respectively, demonstrating good ELF exposure and excellent cell penetration. Saliva concentrations displayed a linear relationship with plasma concentrations (R2=0.76). The ratio of saliva AUC to unbound plasma AUC was close to unity. Gepotidacin exposure in uUTI patients urine (AUC[0-tau]) was high (3742 μg·h/mL on Day 1; 5973 μg·h/mL on Day 4). Urine Ctau exposures ranged from 322 to 352 μg/mL from Day 3 onwards. The minimum gepotidacin urine concentrations remained above a MIC of 4 μg/mL over the 12-hour dosing interval. Gepotidacin renal excretion for uUTI participants was higher than healthy subjects (20% versus 7.5% of dose). Gepotidacin free drug concentrations were measured from swabs collected in women with acute uUTI.

In conclusion, gepotidacin demonstrates favourable distribution characteristics, such as enhanced intracellular penetration, ELF levels superior to and saliva exposures similar to plasma. In addition, high urinary exposures cover MIC values of interest for treating UTI.

### Example 3: Phase II Study Evaluating Gepotidacin in the Treatment of Uncomplicated Urinary Tract Infections

Methods:

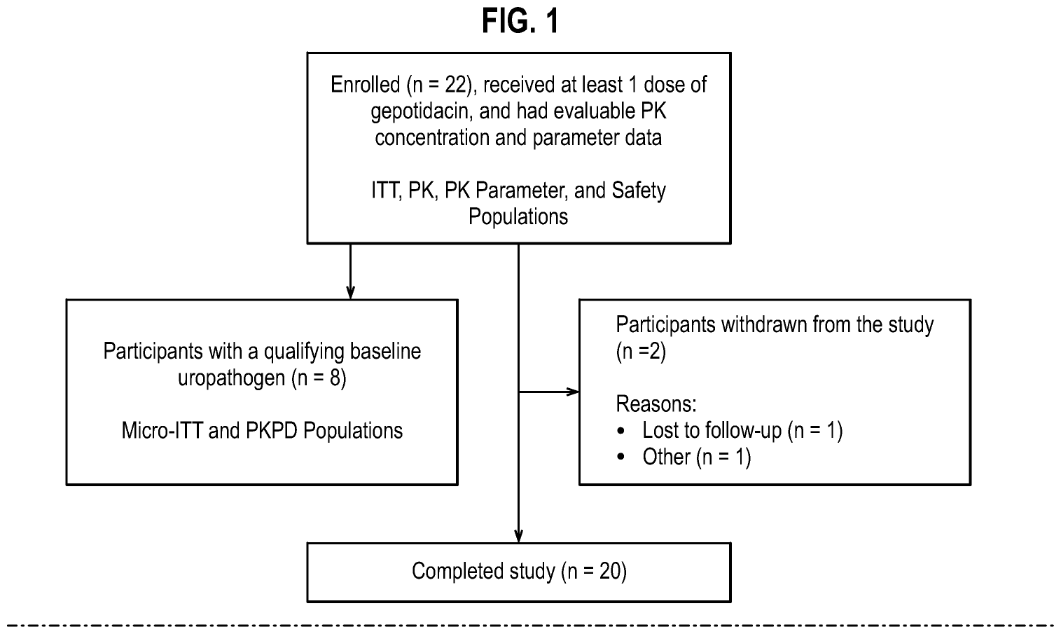

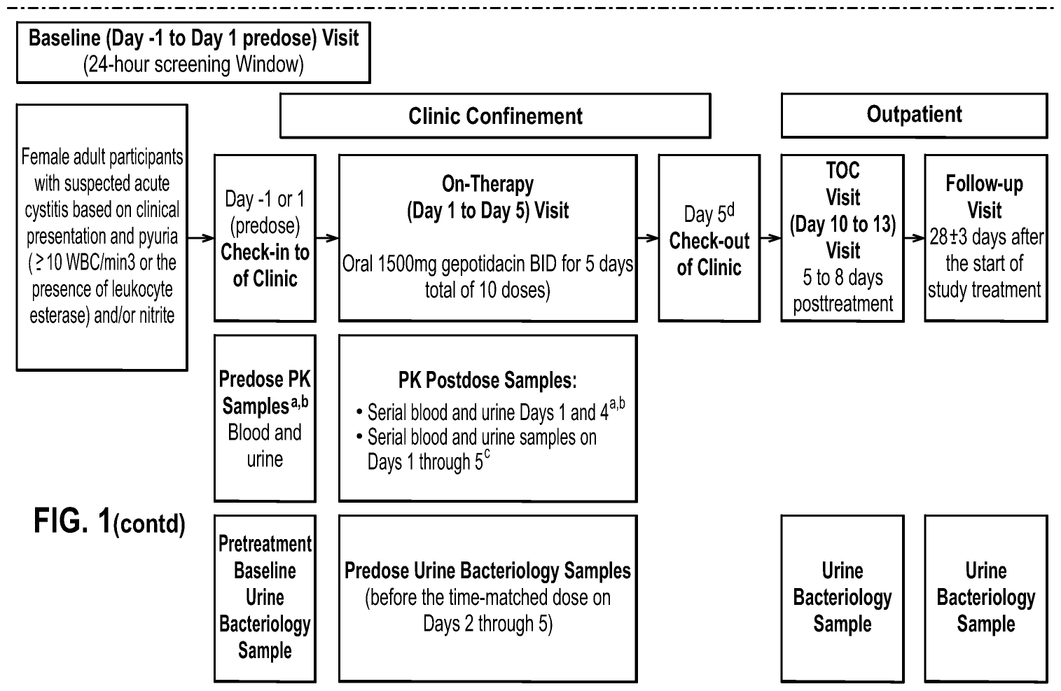

This phase IIa single center study evaluated the safety, tolerability, pharmacokinetics, and efficacy of oral gepotidacin 1500 mg BID for 5 days in female subjects with acute cystitis. Clean catch mid-stream urine specimens were obtained for quantitative culture by standard methods. Urine samples were taken from all participants pre-treatment (baseline) and at all post-baseline visits. All urine samples were sent to a central laboratory (PPD Global Clinical Laboratories, Highland Heights, Ky., USA) for Gram stain, quantitative culture, pathogen identification and susceptibility testing. See FIG. 1 for participant disposition and study outline.

A. Microbiological Studies

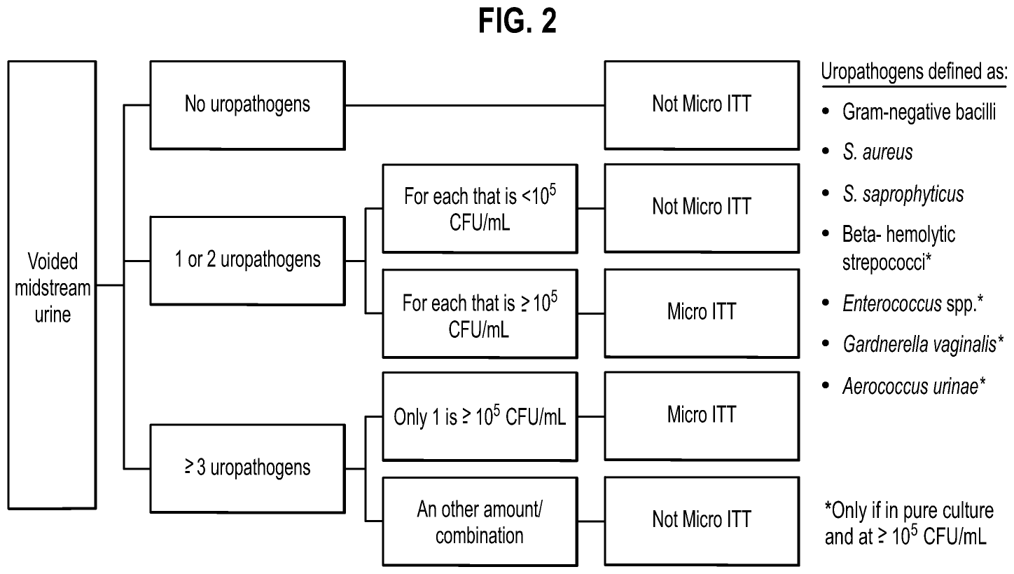

Susceptibility testing by CLSI broth microdilution and gradient diffusion (fosfomycin only) was conducted (CLSI, 2015, Methods for dilution antimicrobial susceptibility tests for bacteria that grow aerobically; approved standard-tenth edition; and CLSI, 2018. Performance Standards for Antimicrobial Susceptibility Testing; Twenty-seventh Informational Supplement M100-S28). Inclusion in the microbiological intent-to-treat population (micro-ITT), required growth of a qualifying baseline uropathogen (≥10⁵ CFU/mL). See FIG. 2 for the baseline algorithm. Microbiological success was defined as culture confirmed eradication (no growth, <10³ CFU/mL) of the qualifying baseline uropathogen and was determined at the Test of Cure (TOC; Day 10-13) and Follow-up Visits (Day 28).

For the purpose of this study, multidrug-resistance (MDR) was defined as a uropathogen that was resistant to ≥3 relevant antibiotic classes; and extended spectrum beta-lactamase (ESBL) production was defined as a E. coli and K. pneumoniae uropathogen with a ceftazidime, aztreonam, cefotaxime or ceftriaxone MIC ≥2 μg/mL.

Results:

Of 22 participants, 8 (36%) had a baseline qualifying uropathogen (5 E. coil, 1 S. saprophyticus, 1 K. pneumoniae, and 1 C. koseri) and were included in the micro-ITT. 14 participants (64%) did not have a baseline qualifying uropathogen (Table 3).

TABLE 3
Summary of Disease Characteristics at Baseline - Qualifying
Uropathogens Recovered (Intent-to-Treat Population)

| | Total N = 22 |
|---|---|
| Number of Participants | n (%) |
| Participants with a qualifying uropathogen | 8 (36) |
| Citrobacter koseri | 1 (13) |
| Escherichia coli | 5 (63) |
| Multidrug-resistant E. coli | 2 (25) |
| Quinolone-resistant E. coli | 1 (13) |
| Klebsiella pneumoniae | 1 (13) |
| Staphylococcus saprophyticus | 1 (13) |

*Only 1 uropathogen was recovered from each participant.

Gepotidacin MICs against the 8 qualifying uropathogens ranged from 0.06-4 μg/mL. Two if coil isolates were multidrug-resistant (defined as resistance to ≥3 antibiotic classes) due to resistance to ampicillin, trimethoprim-sulfamethoxazole and ciprofloxacin/levofloxacin or cefazolin. One additional if E. coli isolate was ampicillin-resistant. See Table 4.

TABLE 4
MICs (μg/mL) for Selected Antimicrobials against E. coli
(Micro-ITT Population, Baseline visit)

| | Participant # (Uropathogen) MIC (μg/mL) | | | | |
|---|---|---|---|---|---|
| | #2 | #3 | #4 | #5 | #6 |
| Compound | (E. coli) | (E. coli) | (E. coli) | (E. coli ^{a}) | (E. coli ^{b}) |
| Ampicillin | >64^{b} | 2 | 1 | >64^{b} | >64^{b} |
| Cefazolin | 2 | 4 | 2 | 4 | 32^{b} |
| Ciprofloxacin | 0.03 | 0.06 | 0.015 | >4^{b} | 0.03 |
| Trimethoprim- | 0.03 | 0.03 | 0.03 | >8^{b} | >8^{b} |
| sulfamethoxazole | | | | | |

^{a} MDR and fluoroquinolone-resistant; b. MDR
^{b} Resistance as defined by M100-S29 CLSI breakpoints
All other results show susceptibility as defined by by M100-S29 CLSI breakpoints.

Microbiological Response for Qualifying Uropathogens

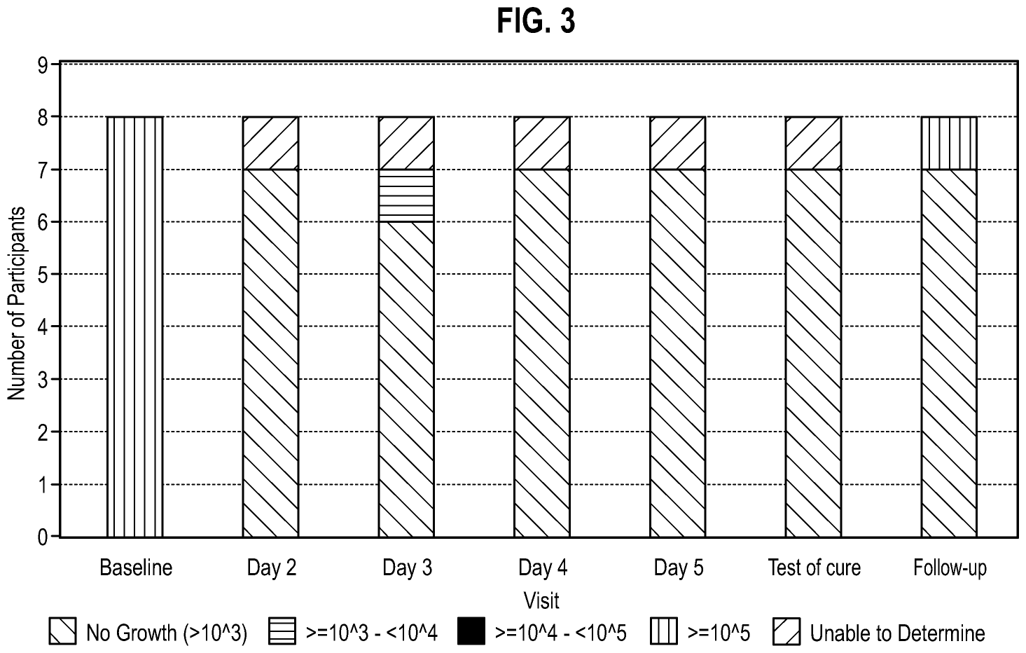

Microbiological response at the Test of Cure (TOC) and Follow up Visits is shown in Table 5. The one microbiological failure at TOC (E. coli) was due to an unreportable (out of stability) urine specimen rather than persistent growth of the uropathogen. In the micro-ITT Population, growth was only observed for 2 isolates posttreatment, including 1 if coil isolate on Day 3 and 1 C. koseri isolate at Follow-up (FIG. 3 shows quantitative bacterial counts (CFU/mL) by baseline qualifying uropathogen over time (micro-ITT population). Of the 8 participants in the micro-ITT, 7 (88%) and 6 (75%) were microbiological successes at the TOC and Follow up Visits, respectively.

TABLE 5
Summary of Microbiological Response at the TOC and
Follow-up Visits (Micro-ITT Population)

| | Microbiological Response, n (%) | |
|---|---|---|
| All Qualifying | (95% Confidence Interval) | |
| Uropathogens | TOC Visit | Follow-up Visit |
| n | 8 | 8 |
| Microbiological Success | 7 (88) (47->99) | 6 (75) (35-97) |
| Microbiological Failure^{a} | 1 (13) (<1-53) | 2 (25) (3-65) |

^{a}Participants considered microbiological failures at the TOC Visit were considered microbiological failures at the Follow-up Visit. A microbiological outcome of unable to determine was considered a microbiological failure.

Antimicrobial Susceptibility of Qualifying Uropathogens

The 2 qualifying other gram-negative uropathogens (C. koseri and K. pneumoniae) were both resistant to ampicillin and were susceptible to all other antimicrobials tested. The 1 qualifying S. saprophyticus uropathogen was susceptible to all antibacterials tested. None of the baseline uropathogens recovered were resistant to nitrofurantoin, fosfomycin, piperacillin/tazobactam or meropenem. No phenotypic ESBL-producing uropathogens were recovered. There was no evidence of reduction in susceptibility to gepotidacin (defined as a ≥4-fold increase in MIC) between a uropathogen obtained at baseline and the same uropathogen at subsequent visits.

For the 4 participants with available steady state PK, qualifying Enterobacteriaceae uropathogens and who were microbiological successes at TOC, plasma fAUC24h/MICs ranged from 7 to 90.5 and urine AUC24h/MICs from 1292 to 121,698. The participant with the lowest plasma fAUC/MIC (7) and urine AUC24h/MIC (1292) had a K. pneumoniae with a gepotidacin MIC of 4 μg/mL.

In addition to the qualifying uropathogens above, gepotidacin MIC was also determined for the two baseline uropathogens which did not qualify for inclusion in the microbiology intent to treat population due to being recovered at lower bacterial counts (<10⁵ CFU/mL): Acinetobacter pittii(MIC 1 mg/L) and Citrobacter freundii complex (MIC 1 mg/L).

PK/PD:

For the 4 participants with available steady state PK, qualifying Enterobacteriaceae uropathogens and who were microbiological successes at TOC, plasma fAUC24h/MICs ranged from 6.99-90.5 and urine AUC24h/MICs from 1292 to 121,698 (Table 6). The participant with the lowest plasma fAUC/MIC (6.99) and urine AUC24h/MIC (1292) had a K. pneumoniae with a gepotidacin MIC of 4 μg/mL and was a microbiological success (Table 6).

TABLE 6
Summary of Plasma and Urine PK/PD and Microbiological
Response at the TOC and Follow-up Visits by Qualifying Uropathogen Isolated at
Baseline (Micro-ITT Population)

| | | | | | Microbiological | |
|---|---|---|---|---|---|---|
| | | GEP MIC | Plasma | Urine | success at | |
| Participant # | Uropathogen | (μg/mL) | fAUC24h/MIC | AUC24h/MIC | TOC | Follow-up |
| 1 | C. koseri | 0.5 | 79.6 | NA | Success | Failure |
| 2 | E. coli | 2 | 22.1 | 7379 | Success | Success |
| 3 | E. coli | 0.5 | 90.5 | 121698 | Success | Success |
| 4 | E. coli | 2 | 30.6 | 9011 | Success | Success |
| 5 | E. colia | 1 | 30.6 | 7926 | Failure^{b} | Failure^{c} |
| 6 | E. colid | 2 | NA | NA | Success | Success |
| 7 | K pneumoniae | 4 | 6.99 | 1292 | Success | Success |
| 8 | S. saprophyticus | 0.06 | 1040 | 543252 | Success | Success |

^{a}MDR and fluoroquinolone-resistant
^{b}Failure due to an out of stability urine specimen
^{c}Microbiological failures at the TOC Visit were considered microbiological failures at the Follow-up Visit
^{d}MDR
NA = Not available (steady state PK was not available);
GEP = gepotidacin

Conclusions

In the micro-ITT Population, microbiological success was achieved in 7 of 8 participants (88%) and 6 of 8 participants (75%) at TOC and Follow-up, respectively. Gepotidacin MIC values ranged from 0.06 to 4 μg/mL against all baseline uropathogens recovered. No participants developed post-treatment uropathogens with reduced susceptibility to gepotidacin (i.e., ≥4-fold increase in gepotidacin MIC).

B. Clinical Efficacy and Safety Analysis of the Phase IIa Study

Methods:

Serial blood and urine PK sampling were performed for the first dose of study treatment on Day 1 and for the time-matched dose on Day 4. Participants took all doses of gepotidacin with food and remained in the clinic to complete a total of 10 doses. Participants were instructed to return for the TOC (Day 10 to 13) and Follow-up (Day 28±3) Visits.

Results:

Summary of Exploratory Endpoints (ITT Population).

Clinical Efficacy: All subjects had significant improvement of clinical symptoms (dysuria, frequency, urgency, lower abdominal pain) within 24 to 48 hrs of treatment. Most subjects, (20/22; 90.9%) achieved symptom resolution at Test of Cure (ToC) and Follow-Up (F/U). Microbiological eradication was achieved independent of baseline CFU's.

Safety Endpoint: Most common AEs involved the GI tract (diarrhoea (18/22 [82%] & nausea 17/22 [77%]). Per investigator observation, tolerance to nausea was observed with repeat dosing. No withdrawal due to AE. There were no clinically relevant trends in safety labs, ECG or vital signs.

Additional Information

Results

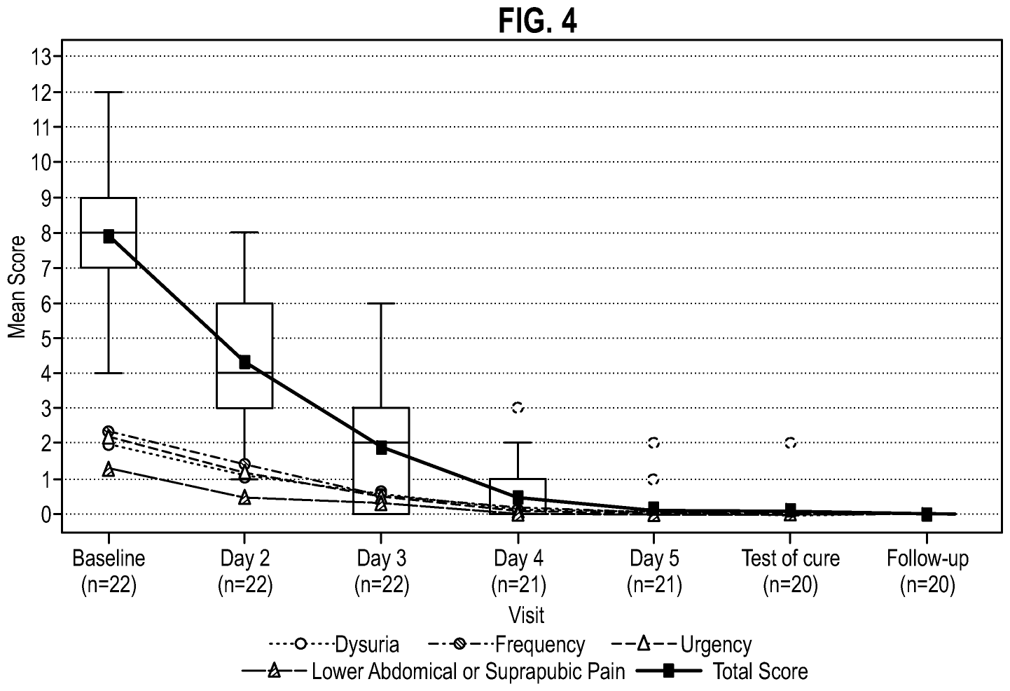

See FIG. 4 for individual clinical symptom score and boxplot of total score over time (intent-to-treat population) Note: The box represents the 25% to 75% percentiles. Within the box, the horizontal line is the median and the square dot is the mean. The upper and lower whiskers represent 1.5× the interquartile range. The open circles represent individual participant outlier scores.

The mean total clinical symptom score in the ITT Population at Baseline was 7.9 (range: 4 to 12) for 4 categories of acute cystitis symptoms consisting of dysuria, frequency, urgency, and lower abdominal or suprapubic pain (score range for each category was 0 to 3). Of the 22 participants enrolled in this Phase IIa study, 19 (86%) and 18 participants (82%) achieved a clinical response of clinical success at the TOC and Follow-up Visits, respectively. At TOC, symptom resolution (i.e., clinical signs and symptom score of 0) was achieved in 19 participants (1 participant had withdrawn from study; 1 participant had no score reported, and 1 participant with incomplete dosing [6 doses] had a score of 2). At Follow-up symptom resolution was achieved in 20 participants (2 participants had withdrawn from study). Lower abdominal or suprapubic pain at Baseline was the most variable symptom category with half of the participants reporting low scores of 0 (27%) or 1 (23%) and the other half of participants reporting high scores of 2 (41%) or 3 (9%). For the other symptom categories, the majority of participants scored a 2.

TABLE 7
Summary of Adverse Events >5% by System Organ
Class and Preferred Term

| System Organ Class | Total |
|---|---|
| Preferred Term | N = 22 n (%) |
| Any adverse event | 21 (95) |
| Gastrointestinal disorders | 21 (95) |
| Diarrhea | 18 (82) |
| Nausea | 17 (77) |
| Vomiting | 5 (23) |
| Infections and infestations | 6 (27) |
| Viral upper respiratory tract infection | 2 (9) |
| Vulvovaginal mycotic infection | 2 (9) |
| Nervous system disorders | 5 (23) |
| Headache | 5 (23) |
| Musculoskeletal and connective tissue disorders | 3 (14) |
| Back pain | 2 (9) |
| General disorders and administration site | 2 (9) |
| conditions | |
| Chest discomfort | 2 (9) |

Participants were housed for the duration of the dosing period, the most frequent adverse events involved the gastro-intestinal tract; all GI AEs were mild or moderate in intensity with onset at day 1, improving upon repeat dosing. Episodes of vomiting were considered drug related in 4 participants, mild or moderate, and not treatment limiting. Use of antiemetics was uncommon and of short duration. The safety profile of gepotidacin was similar to that observed in previous studies with gastrointestinal events, mainly mild or moderate in severity, reported as the most frequent AEs.

### Conclusions

Safety: Overall, oral gepotidacin administered at a dose of 1500 mg BID for 5 days was tolerated with no dose-limiting AEs. The most reported AEs involved the gastrointestinal tract (e.g., primarily diarrhea and nausea). Per investigator observation, tolerance to nausea was observed with repeat dosing. There were no withdrawals or study treatment discontinuations due to AEs. There were no clinically significant changes in safety laboratory parameters, vital signs or ECG intervals.

C. Plasma And Urine Pharmacokinetics

Methods:

For pharmacokinetic assessments, serial blood and urine samples for the assessment of gepotidacin PK concentrations were collected out to 12 hours post dose (T) following the morning dose on Days 1 and 4. CT (trough) concentrations were collected on Days 1-5. PK concentrations of gepotidacin in plasma and urine were measured using a validated LC-MS/MS bio-analytical assay.

Results:

Gepotidacin was rapidly absorbed with median Tmax values of 1.50 to 1.92 hours. Steady state was attained by Day 3 with moderate accumulation in plasma following BID dosing (1.4-fold), which is consistent with an effective elimination half-life of 6.6 hours. Steady-state urine trough levels were high and remained above a minimum inhibitory concentration (MIC) of 4 μg/mL over 12 hours. Approximately 20% of the dose was excreted in urine over the 12-hour dosing interval on Day 1, which increased to 31% on Day 4. Urinary AUC(0-24) (11,945 μg·hr/mL) was higher than the free plasma AUC(0-24) (39.4 μg·hr/mL). Slightly higher gepotidacin plasma and urine exposures were observed in uUTI patients compared to Phase I healthy subjects.

Plasma

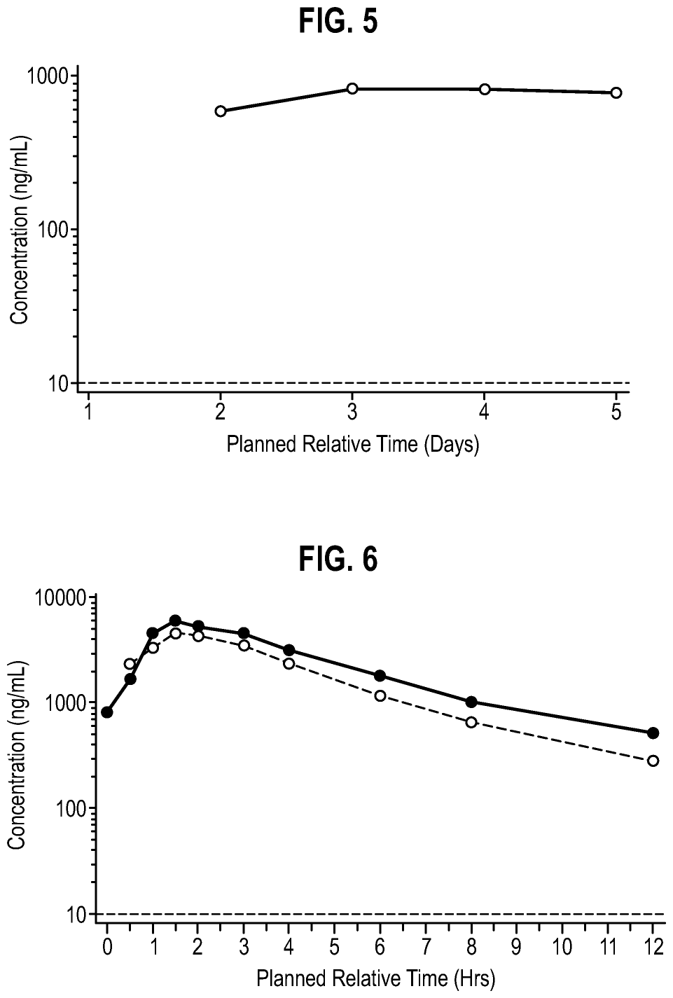

Following repeat twice daily oral administration of 1500 mg, plasma concentrations for gepotidacin peaked rapidly with median Tmax values of 1.50 and 1.92 hours (Days 1 and 4, respectively) and declined in a multiphasic manner. Based on C_{T} (trough) plasma concentrations, steady state was achieved by Day 3. Moderate 1.4-fold accumulation of gepotidacin was observed based on AUC, which was consistent with an effective elimination half-life of 6.6 hours. See FIG. 5 for gepotidacin median C_{T} plasma concentration by day following BID oral administration of gepotidacin (1500 mg), and FIG. 6 for gepotidacin median plasma concentration-time profiles following single and BID oral administration (1500 mg).

TABLE 8
Geometric Mean (% CVb) Estimates for
Key Gepotidacin Plasma PK Parameters

| Parameter (unit) | Day 1 | Day 4 |
|---|---|---|
| AUC(0-T) (μg · hr/mL) | 20.2 (28.6) [n = 20] | 29.3 (31.8) [n = 21] |
| Cmax (μg/mL) | 5.89 (47.3) [n = 20] | 8.44 (38.0) [n = 21] |
| CT (μg/mL) | | 0.851 (41.1) [n = 21] |

Urine

Following repeat twice daily oral administration of 1500 mg gepotidacin, steady-state urine trough concentrations were high and remained above a MIC of 4 μg/mL over the 12-hour dosing interval. Approximately 20% of the dose of gepotidacin was excreted in urine over the 12-hour dosing interval on Day 1, increasing to 31% on Day 4. Steady-state urinary AUC(0-24) (11,945 μg·hr/mL) for gepotidacin was higher than free plasma AUC(0-24) (39.3 μg·h/mL) on Day 4.

TABLE 9
Geometric Mean (% CVb) Estimates for
Key Gepotidacin Urine PK Parameters

| Parameter (unit) | Day 1 | Day 4 |
|---|---|---|
| AUC(0-T) (μg · hr/mL) | 3742 (93.9) [n = 16] | 5973 (87.2) [n = 18] |
| CT (μg/mL) | — | 327 (248.7) [n = 21] |

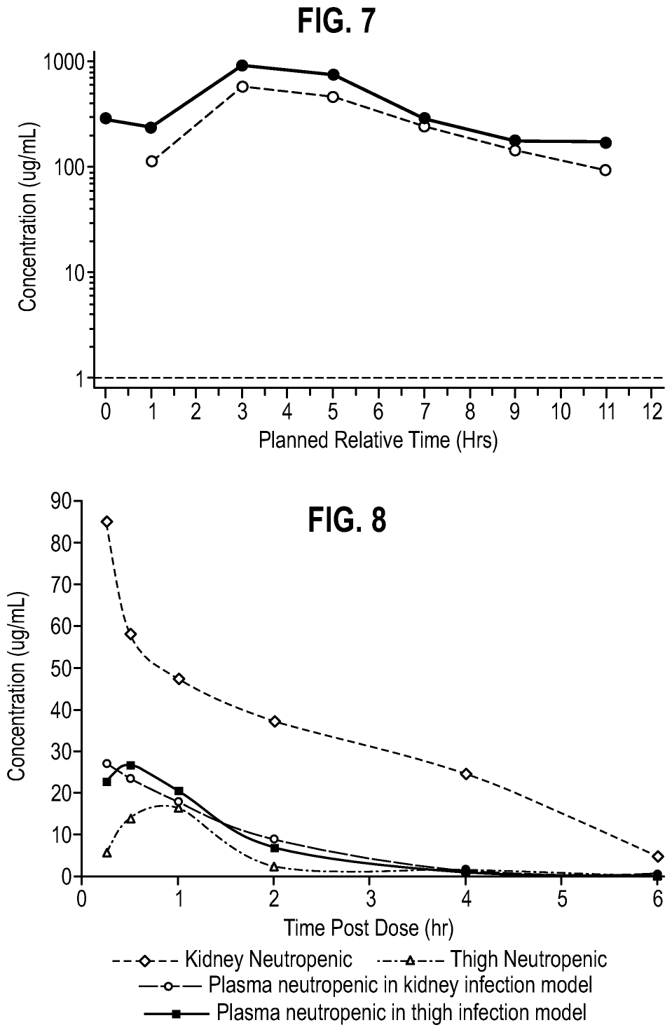

See FIG. 7 for median urine concentration time profiles following single and BID oral administration of gepotidacin (1500 mg).

PK/PD

Following repeat twice daily oral administration of 1500 mg, the mean gepotidacin urine AUC(0-24)/MIC ratio (15,914) was higher than the gepotidacin free plasma AUC(0-24)/MIC ratio (37.0) in the 4 participants with a qualifying E. coli uropathogen at the Baseline visit.

Conclusions

Steady-state gepotidacin plasma exposure was attained by Day 3. There was moderate (1.4-fold) accumulation of gepotidacin in plasma following BID administration. Steady-state urine gepotidacin exposures (AUC[0-24]) exceeded free plasma exposures by approximately 300-fold. Urine concentrations were also higher than the gepotidacin MIC₉₀ values for common uUTI pathogens, such as E. coli (MIC₉₀=4 μg/mL). Given that the bladder is the primary site of infection in acute cystitis, this supports the use of gepotidacin for the treatment of UTI as set out in the present invention. The efficacy of gepotidacin demonstrated in this Phase IIa study provides further support.

### Example 4: Pharmacokinetics-Pharmacodynamics (PK-PD) of Gepotidacin Against in Murine Pyelonephritis and Thigh Infection Models

The objective of this study was to characterize the pharmacokinetic-pharmacodynamic (PK/PD) relationships of gepotidacin in neutropenic mouse thigh and pyelonephritis models against 5 isolates of E. coli covering the range of MICs (1 to 4 μg/mL) to support potential dose selection for urinary tract infection indications.

Methods:

PK and PD studies using gepotidacin were conducted in murine (male CD-1 mice) thigh and kidney infections. The administered doses ranged from 1 to 200 mg/kg SC every 6 hours starting 1h post infection. Infected tissues were evaluated for bacterial burden at 24 h post infection (baseline controls at 1 h post infection).

Plasma and tissue samples (kidney or thigh homogenates) were collected at 15, 30, 60, 120, 240 and 360 minutes. A population PK (PopPK) model was built in NONMEM using plasma exposures.

Efficacy was determined against E. coli ALL, 997577, ATCC25922, IR5 and NCTC13441 (MICs of 1 to 4 μg/mL) in thigh-infected neutropenic (I−) mice and against E. coli ALL in kidney-infected immunocompetent (I+) and I− mice. The PopPK model was used to determine GEP exposures associated with efficacy. PK-PD analyses were conducted using Phoenix WinNonLin 6.3 (Pharsight). The change in login colony forming units (CFU) from baseline were correlated with free drug (f) AUC:MIC using an inhibitory model from the Phoenix library, and model parameter values for each isolate were used to calculate the plasma fAUC:MIC associated with stasis, 1- or 2-login reductions in CFU.

Results:

Plasma PK data were best fit by a 1-compartment IV model with 1St order elimination and were similar in I+vs. I− and thigh- vs. kidney-infected mice.

The AUC₀₋₆ of gepotidacin in kidney was approximately 4- to 5-fold higher than in plasma while the AUC₀₋₆ in thigh was approximately half of plasma.

In the thigh model, median plasma fAUC:MIC ratios for stasis, 1- or 2-log₁₀ reductions in CFU were 11, 16, and 25 (ranges 3-17, 4-25 and 7-40), respectively. Efficacy vs. E. coli ALL was similar in I− mice infected in thigh or kidney. In I+mice, the PK-PD target was reduced by half.

Conclusion:

Median plasma fAUC:MIC targets ranged from 11 to 25. Higher drug levels in kidney vs. plasma or thigh did not translate into improved efficacy in pyelonephritis vs. thigh-infection models.

### Additional Information Example 4

Methods

Pharmacokinetic Studies

Specific pathogen free male CD-1 mice weighing ˜27 g were used throughout the PK and PD studies. For most studies, mice were rendered neutropenic with two IP doses of cyclophosphamide on day −4 (150 mg/kg) and day −1 (100 mg/kg). Plasma and tissue samples (kidney and/or thigh) were collected at 15, 30, 60, 120, 240 and 360 minutes post dose from competent or neutropenic infected mice (N=3/group) following a single subcutaneous (SC) dose of 6.25-200 mg/kg. Samples were assayed by LC/MS/MS; the lower limit of quantification was 0.05 ug/mL.

Pharmacodynamic Studies

PD studies were conducted using thigh and/or kidney infection models in neutropenic or competent mice (N=5/group). Mice were infected with a bacterial suspension in log phase: 100 μL in the left thigh muscle or 50 μL in each of both kidneys (100 μl total). Final inocula across all isolates were 6.0 to 7.0 login CFU/mouse. Dose-ranging studies were conducted with 5 isolates (see Table 1). Starting 1 hr after infection, GEP was given SC (0.2 mL/mouse) at discrete doses of 1-200 mg/kg every 6 hours (q6) for a 24-hour period (4 doses in total). Mice were euthanized 24 hr after start of therapy (6 hr after the last dose), and infected thighs or kidneys were processed to determine viable bacterial counts (CFU). Baseline CFU were obtained from untreated mice at 1 hr post infection and growth control CFU from saline-treated mice at 24 hr.

Data Analysis:

Non-compartmental Analysis (NCA): plasma NCA was conducted using Phoenix WinNonLin 6.3 (Pharsight) with the linear up log down method.

Population Pharmacokinetics (PopPK):
Total drug concentrations in plasma were converted to free drug values based on protein binding of 24% in mice.
A PopPK model was built to describe the drug exposure in plasma over time using NONMEM (7.3) and the software R (version 3.4.0) for diagnostic plots.
The final model was used to simulate exposures at the multiple dose levels evaluated in the efficacy studies.

NCA analyses were conducted with the simulated data. Next, the PK/PD parameters were calculated for drug exposure over 24 hr: Free drug area under the curve over the MIC (fAUC/MIC), time over which free drug concentrations remained above the MIC value (fT>MIC) and free drug Cmax value over the MIC (fCmax/MIC) were obtained.

PK-PD analysis was conducted using Phoenix WinNonLin 6.3 (Pharsight):
Log₁₀ colony forming units (CFU) from each group were correlated with PK-PD parameters using several inhibitory models from the Phoenix library.
Model parameter values for each isolate were used to calculate the PK-PD parameter value associated with stasis, 1-log or 2-log reductions from baseline 1 h controls.

Results

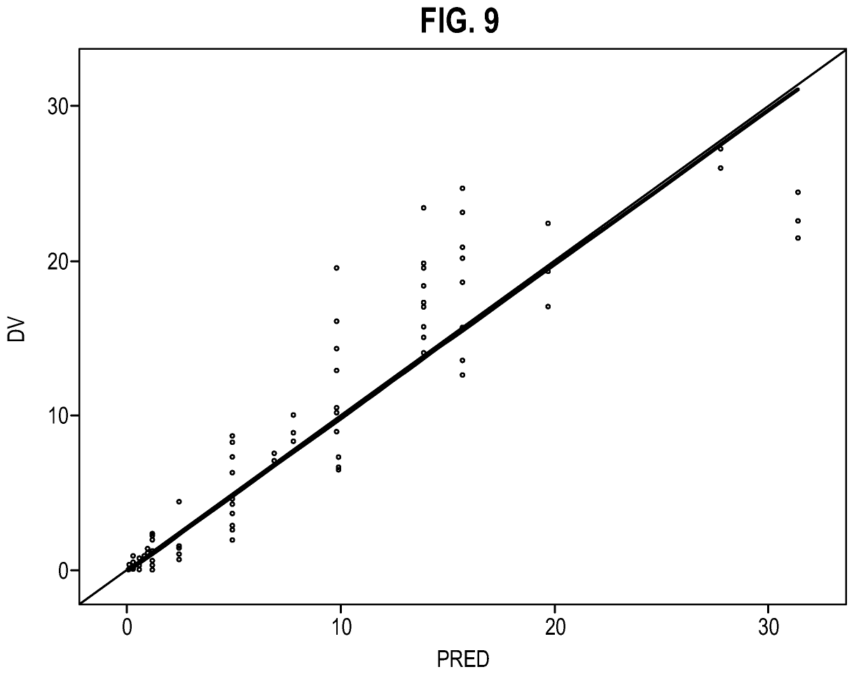

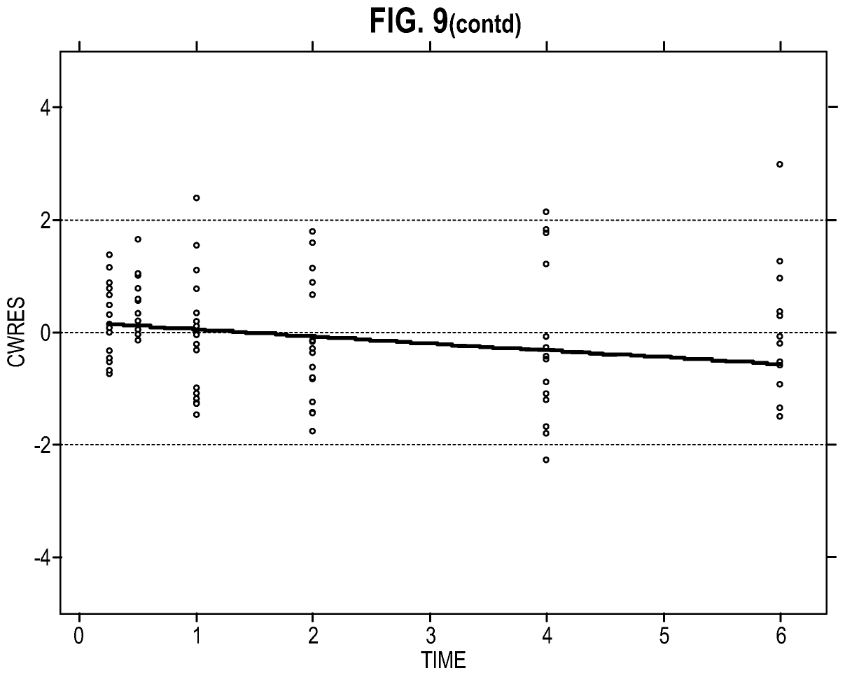

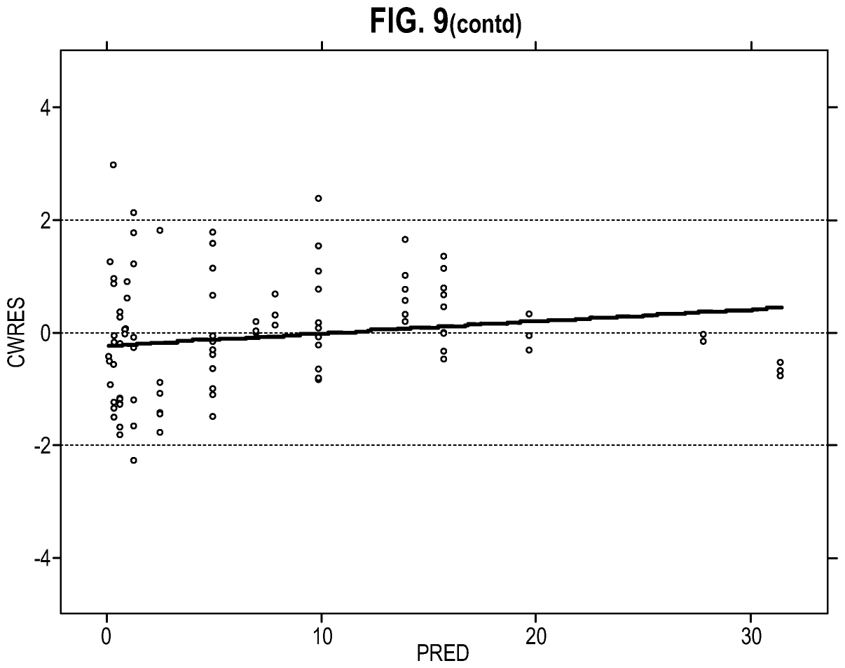

Gepotidacin concentrations were higher in kidney than in plasma or thigh homogenates (see FIG. 8—plasma, kidney and thigh concentration vs. time profiles). AUC and Cmax were approximately dose proportional. No significant difference on the PK of neutropenic vs immunocompetent animals. The PopPK model that best described the gepotidacin disposition in plasma was a one compartment intravenous absorption model with first order elimination, using a combined error model. The final parameter estimates were clearance of 0.104 L/h, volume of distribution of 0.151 L and duration of infusion of 0.262 h (parameter values in agreement with the NCA), proportional error of 41% and additive error of 14.6 μg/mL. Diagnostic plots (FIG. 9: Dependent Variable versus Prediction, and Conditionally Weighted Residuals, versus Time and Prediction) indicate the appropriateness of the developed model.

The inhibitory effect sigmoid Imax model presented the best results on the PK-PD analysis:

$$E = {E}_{0} - \frac{{I}_{max} * PKP D i n d e {x}^{gamma}}{P K P D i n d e {x}^{gamma} + {IC}_{50}^{gamma}}$$

Where E is the change in login CFU at the end of the study compared with baseline controls (ΔCFU); E₀ is the ΔCFU for untreated controls; Iₘₐₓ is the maximum change in CFU between untreated controls and the highest dose tested; IC₅₀ is the PK/PD index value required to produce 50% of the Iₘₐₓ; and gamma is the shape parameter.

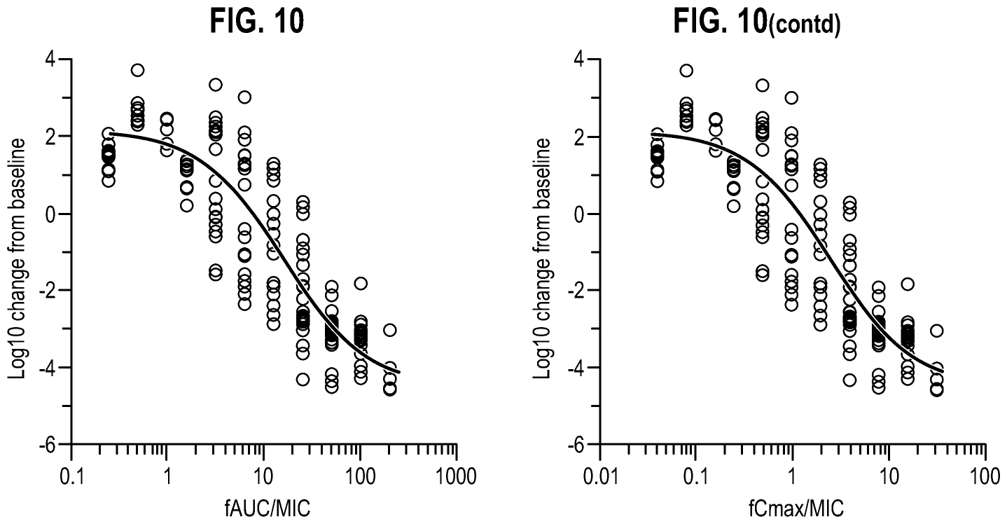

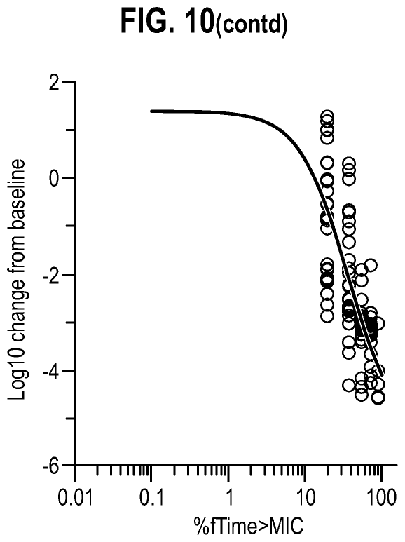

Consistent with previous results obtained from in vitro and in vivo PK/PD studies, fAUC/MIC correlated well with efficacy for gepotidacin when data for all isolates was pooled together (FIG. 10). Tables 10 and 11 display the daily fAUC/MIC in plasma associated with stasis, 1-log or 2-log reductions from baseline across studies:

TABLE 10
Daily fAUC/MIC Ratios for Efficacy in Neutropenic Thigh Model

| | Gepotidacin | | | |
|---|---|---|---|---|
| Strain | MIC | Stasis | 1-log Drop | 2-log Drop |
| ATCC25922 | 1 | 16.8 | 21.5 | 27.6 |
| NCTC13441 | 2 | 16.2 | 24.9 | 40.3 |
| 997577 | 2 | 10.6 | 16.0 | 25.4 |
| ALL | 4 | 3.0 | 6.5 | 13.8 |
| IR5 | 4 | 2.8 | 4.3 | 7.2 |

| | | | |
|---|---|---|---|
| Mean ± SD | 9.9 ± 6.8 | 14.6 ± 9.0 | 22.9 ± 12.8 |
| Median | 10.6 | 16.0 | 25.4 |

TABLE 11
Daily fAUC/MIC Ratios Across Studies with strain “ALL”

| Model | MIC | Stasis | 1-log Drop | 2-log Drop |
|---|---|---|---|---|
| Neutropenic Thigh | 4 | 3.0 | 6.5 | 13.8 |
| Neutropenic UTI | 4 | 4.3 | 8.7 | 15.6 |
| Competent UTI | 4 | 0.7 | 3.3 | 8.1 |

### CONCLUSIONS

Median fAUC/MIC targets were 11, 16 and 25 for stasis, 1-log and 2-log drops, respectively. fAUC/MIC targets were very similar between thigh and kidney infection. fAUC/MIC targets were reduced by approx. half in non-neutropenic mice. Higher drug levels in kidney homogenates vs. plasma or thigh did not translate into improved efficacy in pyelonephritis vs. thigh-infection model.

### Example 5: In Vitro Assays Against Aerobe Organisms

Method 1

Gepotidacin was tested against 101 coagulase-negative staphylococci and 105 viridans streptococci in serial two-fold dilutions and the minimum inhibitory concentration (MIC) was determined as the lowest concentration of the compound that inhibited visible growth.

All of the clinical isolates were collected in 2009-2012 from patient infections from North America, Europe, Latin America and Asia-Pacific medical centers and were mainly obtained from patients with documented nosocomial and community-acquired respiratory tract infections, bloodstream infections and skin and skin structure infections.

Ceftriaxone, meropenem, penicillin, levofloxacin, moxifloxacin and linezolid were included as comparators when testing the viridans streptococci isolates. Oxacillin, levofloxacin, moxifloxacin and linezolid, were included as comparators when testing the coagulase-negative staphylococci isolates. MICs were determined by broth microdilution according to CLSI methods.

The MIC90 for gepotidacin against all the coagulase-negative staphylococci (including S. capitis, S. caprae, S. cohnii, S. epidermidis, S. haemolyticus, S. hominis, S. intermedius, S. simulans and S. warneri) and viridans streptococci (including S. anginosus, S. australis, S. constellatus, S. cristatus, S. gordonii, S. infantarius, S, infantis, S. intermedius, S. massiliensis, S. mitis, S. oralis, S. mutans, S. parasanguinis, S. salivarius, S. sanguinis, and S. vestibularis) isolates tested was 0.5 μg/mL. With the exception of meropenem and moxifloxacin against viridans group streptococci (both with MIC90s of 0.25 μg/mL), this MIC90 was at least 2 to 64-fold lower than the comparators tested.

In addition, the gepotidacin MIC value for Morganella morganii was 4 mg/L, and 8 mg/L for Providencia rettgeri, in each case against at least one isolate tested.

A second study tested gepotidacin by CLSI broth microdilution against bacterial isolates collected from patients with acute bacterial skin and soft tissue infections between 2013-2014.

The MIC90 for gepotidacin against 13 S. epidermidis, 10 S. anginosus and 19 viridans streptococci tested was 0.25, 1 and 0.5 μg/mL, respectively.

Amoxicillin/clavulanic acid, linezolid, fusidic acid, ceftriaxone, ceftaroline, vancomycin, penicillin, quinupristin/dalfopristin, erythromycin, clindamycin, meropenem, tetracycline, chloramphenicol, flucloxacillin, telavancin, daptomycin, trimethoprim/sulfamethoxazole, gentamicin, levofloxacin, tigecycline and cefuroxime were included as comparators. MICs were determined by broth microdilution according to CLSI methods.

In addition, MICs were determined by broth microdilution according CLSI methods and all the comparators from the list above were evaluated against gram-positive aerobe organisms selected from S. lugdenensis, S. agalactiae, Streptococcus group G and Streptococcus group F.

Gepotidacin had a MIC of ≤2 μg/mL against at least one strain of every organism listed above.

Method 2

The compounds were tested in serial two-fold dilutions and the minimum inhibitory concentration (MIC) was determined as the lowest concentration of the compound that inhibited visible growth.

In particular, gepotidacin was tested against 4 strains of Staphylococcus epidermidis from the collection of isolates at Laboratory Specialists, Inc., Westlake, Ohio.

Levofloxacin was included as the comparator in the study to determine the effect of urine on the in vitro activity of gepotidacin against Staphylococcus epidermidis. MICs were determined by broth microdilution according CLSI methods.

The minimum inhibitory concentration (MIC) for gepotidacin was 0.5 μg/mL against 2 isolates of methicillin-susceptible S. epidermidis and the MIC range was 0.25-0.5 μg/mL against 2 isolates of methicillin-resistant S. epidermidis. The MIC value for gepotidacin was 2-fold greater than the levofloxacin MIC for the methicillin-susceptible S. epidermidis and was 4-fold greater than the levofloxacin MIC for each of the methicillin-resistant S. epidermidis.

At least one exemplified salt of gepotidacin was tested (i.e., such as mesylate salt).

Gepotidacin had a MIC of ≤0.5 μg/mL against at least one strain of methicillin-susceptible or methicillin-resistant S. epidermidis.

The data shown in the table below are results from an in vitro study conducted with the same S. epidermidis isolates described above, to determine the effect of urine on the in vitro activity of gepotidacin and levofloxacin against S. epidermidis. Study strains were tested for MICs according to reference CLSI broth microdilution method using cation-adjusted Mueller Hinton Broth (CAMHB) and with the addition of 25%, 50% and 100% urine (not pH adjusted, pH 6.42) and 100% urine (pH adjusted, 7.31 and 8.07). MIC results (mean dilution difference) for gepotidacin were not significantly impacted (mean dilution differences of 0 to −1.01) by the addition of urine. MIC results for levofloxacin were more affected by 100% pooled urine at pH 8.07 with a mean dilution difference of approximately 1.8.

TABLE 12
Comparison of the CLSI MIC broth microdilution reference
method (CAMHB) to urine conditions

| | | | | | |
|---|---|---|---|---|---|
| | | Mean | Mean | | |
| Comparative | Mean | differ- | Dilution | N(%) ± 1 | N(%) ± 2 |
| Condition | MIC | ence | Difference | dilution | dilution |

Staphylococcus epidermidis (n = 4)

| | | | | | |
|---|---|---|---|---|---|
| Gepotidacin | | | | | |
| CAMHB | 0.42 | | | | |
| (ref. method, pH 7.2) | | | | | |
| 25% Urine (pH 7.09) | 0.25 | −0.17 | −0.75 | 4 (100%) | 4 (100%) |
| 50% Urine (6.99) | 0.21 | −0.21 | −1.01 | 4 (100%) | 4 (100%) |
| 100% Urine (pH 6.42) | 0.35 | −0.07 | −0.26 | 4 (100%) | 4 (100%) |
| 100% Urine (pH 7.31) | 0.50 | 0.08 | 0.25 | 4 (100%) | 4 (100%) |
| 100% Urine (pH 8.07) | 0.42 | 0.00 | 0.00 | 4 (100%) | 4 (100%) |
| Levofloxacin | | | | | |
| CAMHB | 0.15 | | | | |
| (ref. method, pH 7.2) | | | | | |
| 25% Urine (pH 7.09) | 0.17 | 0.03 | 0.25 | 4 (100%) | 4 (100%) |
| 50% Urine (6.99) | 0.17 | 0.03 | 0.25 | 4 (100%) | 4 (100%) |
| 100% Urine (pH 6.42) | 0.21 | 0.06 | 0.51 | 4 (100%) | 4 (100%) |
| 100% Urine (pH 7.31) | 0.25 | 0.10 | 0.78 | 3 (75%) | 4 (100%) |
| 100% Urine (pH 8.07) | 0.50 | 0.35 | 1.78 | 2 (50%) | 3 (75%) |

In conclusion, these studies demonstrate the in vitro activity of gepotidacin against the methicillin-susceptible and methicillin-resistant S. epidermidis isolates tested with MICs 40.5 ug/mL, and no significant change in MIC (mean dilution differences of 0 to −0.26) in 100% urine. The fact that gepotidacin activity is not significantly affected by urine makes it a suitable treatment option for UTI.

### Example 6: In Vitro Assays Against Anaerobe Organisms

Studies were conducted to assess the in vitro activity of gepotidacin and the specific comparator compounds as identified in Methods below.

Method 1

Antimicrobial activity was determined by agar dilution using the Clinical and Laboratory Standards Institute (CLSI) recommended procedure.

The compounds were tested in serial two-fold dilutions and the minimum inhibitory concentration (MIC) was determined as the lowest concentration of the compound that inhibited visible growth. Gepotidacin was tested against 333 gram-negative anaerobic and 203 gram-positive anaerobic isolates collected from clinical samples in North America and Europe from 2000 to 2017; the majority collected from 2013 to 2016.

Ceftriaxone, clindamycin, imipenem, metronidazole, moxifloxacin and piperacillin/tazobactam were included as comparators.

The MIC90 (MIC which inhibits 90% of the isolates tested) for gepotidacin was ≤4 μg/mL against the gram-negative and gram-positive anaerobes tested and are shown in the table below.

TABLE 13
Evaluation of gepotidacin Against Gram-negative Anaerobic
Pathogens

| | | Gepotidacin |
|---|---|---|
| Pathogen | Number of Isolates | MIC₉₀ (μg/mL) |
| Bacteroides spp.^{a} | 191 | 4 |
| Bilophila wadsworthia | 26 | 0.5 |
| Fusobacterium spp.^{b} | 25 | 2 |
| Porphyromonas spp.^{c} | 26 | 1 |
| Prevotella spp.^{d} | 30 | 4 |
| Sutterella wadsworthensis | 10 | 1 |
| Veillonella spp.^{e} | 25 | 0.12 |

Key:
^{a}= Bacteroides spp. (n): B. caccae (2), B. fragilis (114), B. ovatus (11), B. stercoris (3), B. thetaiotaomicron (48), B. uniformis (4), B. vulgatus (9).
^{b}= Fusobacterium spp. (n): F. necrophorum (3), F. nucleatum (17), Fusobacterium, non-speciated (5).
^{c}= Porphyromonas spp. (n): P. asaccharolytica (9), P. endodontalis (2), P. gingivalis (2), P. levii (1), P. somerae (5), Porphyromonas, non-speciated (7).
^{d}= Prevotella spp. (n): P. bivia (11), P. buccae (10), P. denticola (5), P. disiens (1), P. melaninogenica (3).
^{e}= Veillonella spp. (n): V. alcalescens dispar (1), V. parvula (9), Veillonella, non-speciated (15).

TABLE 14
Evaluation of Gepotidacin Against Gram-positive Anaerobic Pathogens

| | | Gepotidacin |
|---|---|---|
| Pathogen | Number of Isolates | MIC₉₀ (μg/mL) |
| Bifidobacterium spp.^{a} | 26 | 0.5 |
| Clostridium difficile | 100 | 2 |
| Eggerthella lenta | 21 | 4 |
| Eubacterium spp.^{b} | 31 | 2 |
| Lactobacillus spp.^{c} | 91 | 1 |
| Peptostreptococcus anaerobius | 25 | 0.03 |

^{a}= Bifidobacterium spp. (n): B. adolescentis (5), B. breve (3), B. dentium (4), B. longum (7), B. pseudocatenulatum (3), Bifidobacterium, non-speciated (4).
^{b}= Eubacterium spp. (n): Collinsella (Eubacterium) aerofaciens (5), E. limosum (2), E. nodatum (1), Eubacterium, non-speciated (23).
^{c}= Lactobacillus spp. (n): L. acidophilus (1), L. crispatus (3), L. fermentum (5), L. gasseri (21), L. iners (2), L. jensenii (6), L. plantarum (1), L. rhamnosus (19), Lactobacillus, non-speciated (33).

For all the gram-negative anaerobe organisms combined, gepotidacin MIC90 was 4 μg/mL. This MIC90 value was lower than that for ceftriaxone, clindamycin, moxifloxacin and piperacillin/tazobactam (overall MIC90 values of 512, >8, 8, 16 μg/mL, respectively), and higher than that for imipenem and metronidazole (overall MIC90 values of 0.5, and 2 μg/mL, respectively).

For the gram-positive anaerobe organisms combined, gepotidacin MIC90 was 2 μg/mL. Based on MIC90s, gepotidacin showed increased activity against gram-positive anaerobe organisms compared to ceftriaxone (256 μg/mL), clindamycin (>8 μg/mL), imipenem (8 μg/mL), moxifloxacin (>8 μg/mL) and piperacillin-tazobactam (16 μg/mL), and decreased activity compared to metronidazole (0.5 μg/mL).

A second study tested gepotidacin by CLSI agar dilution against gram-positive and gram-negative anaerobic bacterial isolates from the GlaxoSmithKline Upper Providence culture collection.

The MICs for gepotidacin against 10 Bacteroides spp., 3 Fusobacterium spp., 1 Prevotella spp., 1 Clostridium bifermentans and 4 Peptostreptococcus spp. tested was 0.0.12-16, 0.12-1, 4, 0.06 and 0.12-2 μg/mL, respectively.

Amoxicillin, azithromycin, levofloxacin, and cefuroxime were included as comparators.

Conclusion to Method 1

These studies demonstrate the in vitro activity of gepotidacin against gram-negative anaerobe organism (MIC90=4 μg/mL) and gram-positive anaerobe organisms (MIC90=2 μg/mL) (MIC ≤2 μg/mL against at least one strain of every organism listed).

Additional Data from Method 1

Additional analysis was performed from the results of the study described in Method 1 above, to determine the in vitro activity of gepotidacin against gram-negative and gram-positive anaerobic organisms with resistance to ceftriaxone, clindamycin, imipenem, metronidazole, moxifloxacin and piperacillin/tazobactam.

Table 15 shows the MIC ranges against drug-resistant Bacteroides spp. when tested by agar dilution. MIC₅₀/MIC₉₀ was not calculated due to the low number of isolates (n<10) for the majority of drug-resistant subsets.

TABLE 15
Gepotidacin MICs (μg/mL) against drug-resistant Bacteroides spp.,
tested by agar dilution

| | Number | Gepotidacin |
|---|---|---|
| | of | MIC range |
| Organism/Phenotype (Method) | isolates | (μg/mL) |
| Bacteroides spp. ceftriaxone R (Agar Dilution) | 100 | ≤0.015-32 |
| Bacteroides caccae | 2 | 0.05-8 |
| Bacteroides fragilis | 35 | 0.25-32 |
| Bacteroides ovatus | 8 | 1-4 |
| Bacteroides stercoris | 2 | 0.12-1 |
| Bacteroides thetaiotaomicron | 43 | 0.5-16 |
| Bacteroides uniformis | 3 | 1-2 |
| Bacteroides vulgatus | 7 | ≤0.015-4 |
| Bacteroides spp. clindamycin R- (Agar Dilution) | 53 | ≤0.015-32 |
| Bacteroides caccae | 1 | 8 |
| Bacteroides fragilis | 22 | ≤0.015-32 |
| Bacteroides ovatus | 4 | 1-4 |
| Bacteroides thetaiotaomicron | 21 | 0.5-8 |
| Bacteroides uniformis | 1 | 1 |
| Bacteroides vulgatus | 4 | ≤0.015-2 |
| Bacteroides fragilis imipenem R (Agar Dilution) | 1 | 0.5 |
| Bacteroides spp moxifloxacin R (Agar Dilution) | 32 | 0.25-32 |
| Bacteroides caccae | 1 | 0.5 |
| Bacteroides fragilis | 19 | 0.25-32 |
| Bacteroides ovatus | 2 | 1-4 |
| Bacteroides thetaiotaomicron | 6 | 0.5-8 |
| Bacteroides uniformis | 1 | 1 |
| Bacteroides vulgatus | 3 | 0.25-4 |
| Bacteroides fragilis pip/tazo R (Agar Dilution) | 2 | 0.5-4 |

Table 16 shows the MIC ranges against drug-resistant Bacteroides spp. when tested by broth microdilution. MIC₅₀/MIC₉₀ was not calculated due to the low number of isolates (n<10) for the majority of drug-resistant subsets.

TABLE 16
Gepotidacin MICs (μg/mL) against drug-resistant Bacteroides spp.,
tested by broth microdilution

| | Number | Gepotidacin |
|---|---|---|
| | of | MIC range |
| Organism/Phenotype (Method) | isolates | (μg/mL) |
| Bacteroides spp ceftriaxone R (Broth | 61 | ≤0.015-6 |
| Microdilution) | | |
| Bacteroides caccae | 1 | 0.25 |
| Bacteroides fragilis | 24 | 0.25-16 |
| Bacteroides ovatus | 5 | 0.5-2 |
| Bacteroides stercoris | 2 | ≤0.015-0.5 |
| Bacteroides thetaiotaomicron | 20 | 0.5-8 |
| Bacteroides uniformis | 2 | 0.5-2 |
| Bacteroides vulgatus | 7 | 0.06-4 |
| Bacteroides spp clindamycin R (Broth | 51 | 0.06-16 |
| Microdilution) | | |
| Bacteroides caccae | 1 | 4 |
| Bacteroides fragilis | 23 | 0.06-16 |
| Bacteroides ovatus | 5 | 0.25-4 |
| Bacteroides thetaiotaomicron | 18 | 0.5-8 |
| Bacteroides vulgatus | 4 | 0.06-0.5 |
| Bacteroides fragilis imipenem R (Broth | 2 | 1-4 |
| Microdilution) | | |
| Bacteroides spp moxifloxacin R (Broth | 35 | 0.25-8 |
| Microdilution) | | |
| Bacteroides caccae | 1 | 0.25 |
| Bacteroides fragilis | 20 | 0.25-8 |
| Bacteroides ovatus | 3 | 0.5-4 |
| Bacteroides thetaiotaomicron | 7 | 0.5-8 |
| Bacteroides uniformis | 1 | 0.5 |
| Bacteroides vulgatus | 3 | 0.25-4 |
| Bacteroides fragilis pip/tazo R (Broth | 2 | 1-4 |
| Microdilution) | | |

Table 17 shows the MIC ranges against drug-resistant gram-negative anaerobes (other than Bacteroides spp.) when tested by agar dilution. MIC₅₀/MIC₉₀ was not calculated due to the low number of isolates (n<10) for all drug-resistant subsets.

TABLE 17
Gepotidacin MICs (μg/mL) against drug-resistant gram-negative
anaerobes (other than Bacteroides spp.)., tested by agar dilution

| | Number | Gepotidacin |
|---|---|---|
| | of | MIC range |
| Organism/Phenotype (Method) | isolates | (μg/mL) |
| Fusobacterium non-speciated ceftriaxone R | 1 | 2 |
| Fusobacterium spp. clindamycin R | 2 | 0.25-2 |
| Fusobacterium necrophorum | 1 | 0.25 |
| Fusobacterium, non-speciated | 1 | 2 |
| Porphyromonas non-speciated. ceftriaxone R | 1 | 0.125 |
| Porphyromonas spp. clindamycin R | 4 | 0.06->32 |
| Porphyromonas asaccharolytica | 2 | 0.06->32 |
| Porphyromonas somerae | 1 | 0.06 |
| Porphyromonas, non-speciated | 1 | 0.06 |
| Porphyromonas non-speciated. imipenem R | 1 | 0.125 |
| Porphyromonas levii metronidazole R | 1 | ≤0.015 |
| Prevotella spp. ceftriaxone R | 6 | 0.25-2 |
| Prevotella bivia | 1 | 1 |
| Prevotella buccae | 4 | 0.25-2 |
| Prevotella melaninogenica | 1 | 0.25 |
| Prevotella spp. clindamycin R | 7 | 0.25-4 |
| Prevotella bivia | 2 | 0.5-4 |
| Prevotella buccae | 4 | 0.25-4 |
| Prevotella melaninogenica | 1 | 0.25 |
| Prevotella bivia moxifloxacin R | 2 | 4 |
| Sutterella wadsworthensis metronidazole R | 4 | 0.5-1 |
| Veillonella parvula. ceftriaxone R | 1 | ≤0.015 |
| Veillonella spp. clindamycin R | 5 | 0.125 |
| Veillonella parvula | 1 | 0.125 |
| Veillonella, non-speciated | 4 | 0.125 |
| Veillonella spp. moxifloxacin R | 3 | ≤0.015-0.03 |
| Veillonella parvula | 2 | ≤0.015-0.03 |
| Veillonella, non-speciated | 1 | ≤0.015 |
| Veillonella parvula pip/tazo R | 1 | 0.03 |

Table 18 shows the MIC ranges against drug-resistant gram-positive anaerobes when tested by agar dilution. MIC₅₀/MIC₉₀ was not calculated due to the low number of isolates (n<10) for the majority of drug-resistant subsets.

TABLE 18
Gepotidacin MICs (μg/mL) against drug-resistant gram-positive
anaerobes, tested by agar dilution

| | Number | Gepotidacin |
|---|---|---|
| | of | MIC range |
| Organism/Phenotype (Method) | isolates | (μg/mL) |
| Bifidobacterium pseudocatenulatum. | 1 | 4 |
| clindamycin R | | |
| Bifidobacterium spp. moxifloxacin R | 3 | 0.125-0.5 |
| Bifidobacterium adolescentis | 1 | 0.25 |
| Bifidobacterium breve | 1 | 0.5 |
| Bifidobacterium longum | 1 | 0.12 |
| Clostridioides difficile ceftriaxone R | 48 | 0.5-4 |
| Clostridioides difficile clindamycin R | 23 | 0.5-2 |
| Clostridioides difficile imipenem R | 2 | 1 |
| Clostridioides difficile moxifloxacin R | 37 | 0.5-8 |
| Eggerthella lenta ceftriaxone R | 19 | 0.06-32 |
| Eggerthella lenta clindamycin R | 3 | 1-4 |
| Eggerthella lenta moxifloxacin R | 6 | 1-32 |
| Eubacterium non-speciated ceftriaxone R | 1 | 2 |
| Eubacterium non-speciated. clindamycin R | 1 | 2 |
| Eubacterium spp. moxifloxacin R | 3 | 0.25-2 |
| Eubacterium limosum | 2 | 0.25-0.5 |
| Eubacterium, non-speciated | 1 | 2 |
| Eubacterium nodatum. metronidazole R | 1 | 0.125 |
| Peptostreptococcus anaerobius clindamycin R | 3 | ≤0.015-0.03 |
| Peptostreptococcus anaerobius moxifloxacin R | 3 | 0.03 |
| Lactobacillus spp. clindamycin R | 3 | 0.03-1 |
| Lactobacillus gasseri | 2 | 0.5-1 |
| Lactobacillus, non-speciated | 1 | 0.03 |
| Lactobacillus spp. imipenem R | 27 | 0.03-1 |
| Lactobacillus rhamnosus | 18 | 0.06-1 |
| Lactobacillus, non-speciated | 9 | 0.03-1 |

Conclusion to Additional Data from Method 1

This study demonstrates the in vitro activity of gepotidacin against drug-resistant gram-negative and gram-positive anaerobe organisms with MICs ≤4 μg/mL against at least one strain of every drug-resistant phenotype listed in the tables above, with the exception of 1 isolate of Bacteroides caccae which had a MIC=8 mg/L, when tested by agar dilution.

### Example 7: In Vitro Assays Against Gram Negative Aerobe Organisms

Gepotidacin was tested against the following organisms obtained from UTI patients, in serial two-fold dilutions. The minimum inhibitory concentration (MIC) was determined by broth microdilution according to CLSI methods.

For Acidovorax temperans (n=1), gepotidacin MIC was 1 μg/mL.

For Citrobacter amalonaticus (n=1), gepotidacin MIC was 2 μg/mL.

For Providencia stuartii (n=1), gepotidacin MIC was 32 μg/mL.

For Pseudomonas putida (n=4), gepotidacin MIC was 8 or 16 μg/mL.

It is to be understood that the invention is not limited to the aspects or embodiments illustrated hereinabove and the right is reserved to the illustrated aspects or embodiments and all modifications coming within the scope of the following claims.

The various references to journals, patents, and other publications which are cited herein comprise the state of the art and are incorporated herein by reference as though fully set forth.

1. A method for treating urinary tract infection (UTI) comprising administering gepotidacin or a pharmaceutically acceptable salt thereof, in a therapeutically effective amount in a human in need thereof, wherein the UTI is caused by one or more bacterium selected from:
Staphylococcus saprophyticus; Acinetobacter baumannii, Acinetobacter baumannii anitratus, Acinetobacter pittii, Citrobacter freundii complex, Citrobacter koseri, Haemophilus parainfluenzae, Haemophilus paraphrophilus, Klebsiella oxytoca, Klebsiella variicola, Leclercia adecarboxylata, Proteus hauseri, Proteus peneri, Serratia marcescens, Shigella boydii, Shigella flexneri, Shigella sonnei, Morganella morganii, Providencia rettgeri, drug-resistant Klebsiella pneumoniae, drug-resistant Escherichia coli, Acidovorax temperans, Citrobacter amalonaticus, Providencia stuartii, Pseudomonas putida;
Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans, Staphylococcus warneri, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massiliensis, Streptococcus mitts, Streptococcus oralis, Streptococcus mutans, Streptococcus parasanguinis, Streptococcus salivarius, Streptococcus sanguinis, Streptococcus vestibularis;
Bilophila wadsworthia, Sutterella wadsworthensis, Clostridium bifermentans, Clostridium difficile, Eggethella lenta, Peptostreptococcus anaerobius, Peptostreptococcus anaerobius, Bacteroides caccae, Bacteroides fragilis, Bacteroides ovatus, Bacteroides stercoris, Bacteroides thetaiotaomicron, Bacteroides uniformis, Bacteroides vulgatus, Fusobacterium necrophorum, Fusobacterium nucleatum, Porphyromonas asaccharolytica, Porphyromonas endodontalis, Porphyromonas gingivalis, Porphyromonas levii, Porphyromonas somerae, Prevotella bivia, Prevotella buccae, Prevotella denticola, Prevotella disiens, Prevotella melaninogenica, Veillonella alcalescens dispar, Veillonella parvula, Bifidobacterium adolescentis, Bifidobacterium breve, Bifidobacterium dentium, Bifidobacterium longum, Bifidobacterium pseudocatenulatum, Collinsella (Eubacterium) aerofaciens, Eubacterium limosum, Eubacterium nodatum, Lactobacillus acidophilus, Lactobacillus crispatus, Lactobacillus fermentum, Lactobacillus gasseri, Lactobacillus iners, Lactobacillus jensenii, Lactobacillus plantarum, and Lactobacillus rhamnosus.

2. A method according to claim 1, wherein, prior to the administration of gepotidacin or a pharmaceutically acceptable salt thereof, the UTI is determined to be caused by one or more bacterium named in claim 1.

3. A method for treating urinary tract infection (UTI) in a human comprising the following steps:
a) determining whether a sample from a human suspected of having UTI contains one or more bacterium selected from:
Staphylococcus saprophyticus; Acinetobacter baumannii, Acinetobacter baumannii anitratus, Acinetobacter pittii, Citrobacter freundii complex, Citrobacter koseri, Haemophilus parainfluenzae, Haemophilus paraphrophilus, Klebsiella oxytoca, Klebsiella variicola, Leclercia adecarboxylata, Proteus hauseri, Proteus peneri, Serratia marcescens, Shigella boydii, Shigella flexneri, Shigella sonnet, Morganella morganii, Providencia rettgeri, drug-resistant Klebsiella pneumoniae, drug-resistant Escherichia coli, Acidovorax temperans, Citrobacter amalonaticus, Providencia stuartii, Pseudomonas putida;
Staphylococcus lugdenensis, Streptococcus agalactiae, Streptococcus group F, Streptococcus group G, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans, Staphylococcus warneri, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massiliensis, Streptococcus mitts, Streptococcus oxalis, Streptococcus mutans, Streptococcus parasanguinis, Streptococcus salivarius, Streptococcus sanguinis, Streptococcus vestibularis;
Bilophila wadsworthia, Sutterella wadsworthensis, Clostridium bifermentans, Clostridium difficile, Eggethella lenta, Peptostreptococcus anaerobius, Peptostreptococcus anaerobius, Bacteroides caccae, Bacteroides fragiiis, Bacteroides ovatus, Bacteroides stercoris, Bacteroides thetaiotaomicron, Bacteroides uniformis, Bacteroides vulgatus, Fusobacterium necrophorum, Fusobacterium nucleatum, Porphyromonas asaccharolytica, Porphyromonas endodontalis, Porphyromonas gingivalis, Porphyromonas levii, Porphyromonas somerae, Prevotella bivia, Prevotella buccae, Prevotella denticola, Prevotella disiens, Prevotella melaninogenica, Veillonella alcalescens dispar, Veillonella parvula, Bifidobacterium adolescentis, Bifidobacterium breve, Bifidobacterium dentium, Bifidobacterium longum, Bifidobacterium pseudocatenulatum, Collinsella (Eubacterium) aerofaciens, Eubacterium limosum, Eubacterium nodatum, Lactobacillus acidophilus Lactobacillus crispatus, Lactobacillus fermentum, Lactobacillus gasseri, Lactobacillus iners, Lactobacillus jensenii, Lactobacillus plantarum, or Lactobacillus rhamnosus;
b) administering gepotidacin or a pharmaceutically acceptable salt thereof in a therapeutically effective amount to the human if one or more of the bacterium is identified in the sample in step (a) and is determined to be the cause of the UTI.

4. A method as claimed in claim 1, wherein the UTI is uncomplicated UTI.

5. A method as claimed in claim 1, wherein the UTI is recurrent uncomplicated UTI.

6. A method as claimed in claim 1, wherein the UTI is complicated UTI.

7. A method as claimed in claim 1, wherein the human is female.

8. A method as claimed in claim 1, wherein the human is male.

9. A method as claimed in claim 1, wherein the human is pregnant, adolescent or paediatric.

10. A method for treating uncomplicated UTI, comprising administering gepotidacin or a pharmaceutically acceptable salt thereof in a therapeutically effective amount in a human in need thereof, wherein the uncomplicated UTI is caused by one or more bacterium selected from: Staphylococcus saprophyticus, drug-resistant Staphylococcus saprophyticus, Proteus hauseri, Proteus peneri, drug-resistant Klebsiella pneumoniae, and drug-resistant Escherichia coli.

11. A method as claimed in claim 10, wherein, prior to the administration of gepotidacin or a pharmaceutically acceptable salt thereof, one or more bacterium selected from Staphylococcus saprophyticus, drug-resistant Staphylococcus saprophyticus, Proteus hauseri, Proteus peneri, drug-resistant Klebsiella pneumoniae, and drug-resistant Escherichia coli is determined to be the cause of the uncomplicated UTI.

12-18. (canceled)

19. A method as claimed in claim 3, wherein the UTI is uncomplicated UTI.

20. A method as claimed in claim 3, wherein the UTI is recurrent uncomplicated UTI.

21. A method as claimed in claim 3, wherein the UTI is complicated UTI.

22. A method as claimed in claim 3, wherein the human is female.

23. A method as claimed in claim 3, wherein the human is male.

24. A method as claimed in claim 3, wherein the human is pregnant, adolescent or paediatric.
